(19) United States
(12) Patent Application Publication
(10) Pub. No.: US 2008/0254017 A1
(43) Pub. Date: Oct. 16, 2008

(54) Methods and compositions for treating symptomes of diseases related to imbalance of essential fatty acids

(75) Inventors: Patricia Kane, Millville, NJ (US); Edward Kane, Millville, NJ (US)

Correspondence Address: LAW OFFICES OF KHALILIAN SIRA, LLC, 9100 PERSIMMON TREE ROAD, POTOMAC, MD, 20854, US

(73) Assignee: BodyBio, Inc., Millville, NJ (US)

(21) Appl. No.: 11/455,502

(22) Filed: Jun. 19, 2006

(51) Int. Cl. A61K 38/44; A61K 31/685; A61P 25/00

(52) U.S. Cl. 424/94.4; 514/77

(57) ABSTRACT

The invention as disclosed herein provides pharmaneutical compositions and methods for treating, ameliorating, or preventing the symptoms of fatty acids imbalance and cell membrane dysfunction. The pharmaneutical compositions of the invention contain in an effective amount a first and a second composition, the first composition comprises an effective amount of one or more phosphatidylcholine formulations and the second composition comprises an effective amount of one or more constituents comprising essential fatty acid supplements, trace minerals, phenylbutyrate, electrolytes, methylating agents, reduced glutathione, or a combination thereof, in a suitable carrier.

### FIELD OF THE INVENTION

This invention relates to the treatment of symptoms of diseases and disorders related to an imbalance of essential fatty acids and cell membrane dysfunction.

### I. BACKGROUND OF THE INVENTION

There are a wide variety of diseases and disorders that are caused by or results from cell membrane dysfunction and imbalance and derangement of fatty acids. Studies on red cell fatty acids of subjects suffering from symptoms of fatty acids imbalance demonstrated that this population has characteristic mild to moderate elevation of red cell very long chain fatty acids (VLCFAs) above C20 (carbon 20) indicating peroxisomal involvement (Kane et al., 1997a, 1997b, 1999, and 2002) and (Foster et al., 2002). Altered peroxisomal function represents cellular membrane disturbance, neurological dysfunction, hepatic derangement in regard to detoxification, the potential for an increase in ceramide production and impaired synthesis of prostaglandins that is a complication or etiology of the autistic spectrum.

Peroxisomes are present in virtually all cells (except for mature erythrocytes), and most prevalent in the liver and kidney. They play a critical role of cellular lipid metabolism in the biosynthesis of fatty acids via β-oxidation. The peroxisome is a primary site of detoxication within the cell. See, for example, Gibson et al. 1993. Peroxisomal disorders are characterized by an accumulation in tissue and body fluids of renegade fatty acids: saturated and mono-unsaturated VLCFAs, odd chain fatty acids, and branched chain fatty acids pristanic and phytanic which are normally degraded within the peroxisome. The accumulation of renegade or VLCFAs may constitute a minor part of overall fatty acid content in red cells, however, peroxisomal deficiency disorders with defects in peroxisomal β-oxidation are deleterious to the brain and CNS (see, for example, McGuinness et al., 1993), reflecting blocked detoxification and methylation pathways and may be characteristic in autism, PDD, seizure disorders, stroke, and states of neurotoxicity.

Derangement of red cell lipids pertaining to suppression of peroxisomal β-oxidation has been observed in children with autistic spectrum disorder (Kane, 1997a). Autistic Spectrum Disorder (ASD) is a neurodevelopmental disorder encompassing pervasive developmental delay (PDD) characterized by abnormalities in social interaction, reasoning, learning, symbolic and imaginative play, delayed and disordered language, sensorimotor skills, and stereotypic behavior. Detailed examination of red cell fatty acids of more than 7000 subjects with autism and PDD has demonstrated that this population has characteristic mild to moderate elevation of red cell very long chain fatty acids (VLCFAs) above C20 (carbon 20) indicating peroxisomal involvement (Kane et al., 1997a, 1997b, 1999, and 2002) and (Foster et al., 2002).

Inherited peroxisomal disorders, such as X-linked adrenoleukodystrophy (X-ALD), have been hallmarked by the work of Hugo and Ann Moser (see, for example, Moser et al., 2005a and 2005b). X-ALD is a neuroinflammatory, demyelinating disease and has a typical clinical onset of 2.75 to 10 years of age presenting with behavioral disturbances, poor school performance, difficulty understanding speech, attention deficit, hyperactivity, deterioration of vision (visual field cuts), impaired auditory discrimination, fatigue, anorexia, diarrhea/constipation, abdominal pain, and vomiting (see, for example, Clayton, 2001). The course of some forms of ALD is relentlessly progressive. The biomarkers for ALD are most notably an accumulation of C24:0 and C26:0 in plasma and tissues. The capacity to degrade VLCFAs occurs in the peroxisome via β-oxidation. Disorders of peroxisomal β-oxidation include defects in acyl-CoA oxidase, D-bifunctional proteins, VLCFA-CoA importer and methylacyl-CoA racemase deficiency. Most notably, accumulation of VLCFAs is associated with a deficiency of fatty acyl-CoA oxidase, the enzyme that catalyses the first step in β-oxidation. A prerequisite for β-oxidation is the activation of fatty acids to their Co-A derivatives (see, for example, Shrago, 1995).

As the accumulation of VLCFAs, which may serve as a substrate to form ceramides, has been clearly established to be deleterious to the brain and CNS (Moser and Moser, 1996a and 1996 b), it is plausible that autism and PDD may mimic pseudo-neonatal adrenoleukodystrophy (see, for example, Araki et al., 1994), atypical ALD, asymptomatic ALD or other variations of ALD as a peroxisomal disorder with enlarged peroxisomes, reduced production of acyl-CoA oxidase, suppressed peroxisomal β-oxidation and a compromised neurological system.

Children and adolescents with peroxisomal involvement may present with symptoms of attention-deficit/hyperactivity disorder (ADHD) psychiatric disorders and adults with multiple sclerosis, sensorimotor polyneuropathy, psychosis or progressive cognitive decline. Females who are carriers may also express neurological difficulties yet not fully express ALD, rather a syndrome of ALD. Simon and colleagues describe a unique familial leukodystrophy with adult onset dementia in a brother and sister whose manifestation of their symptoms was after the age of 30 (Simon et al., 1998). Patients presented with progressive cognitive decline, paucity of speech, limited taught content, blunted affect, motor restlessness, poor judgment, impaired short term memory, incontinence of urine and feces, inattention, perseveration, emotional liability and with progression of the presentation, and nonverbal skills. Extensive laboratory investigation was unrevealing, however, a right frontal brain biopsy showed ‘scattered cortical neurons containing coarse, irregular, densely osmophilic material and round lipid droplets (lipofuscin) described as a leukodystrophy with membrane enclosed glycolipid inclusions.

X-ALD does not compromise cognitive development in neurologically asymptomatic boys (Cox et al., 2006). Children with autism and PDD may parallel this phenomenon in that many express normal intelligence and cognition but have difficulty with social interaction. Other children on the autistic spectrum may have what appears to be normal development in their first years of life only to later regress and lose skills such as speech, eye contact, learning, memory (Bauman and Kemper, 2005).

Steroids were previously suggested for children with autistic spectrum disorder by Chez and colleagues (Lewine et al., 1999) which links directly to disturbances in β-oxidation of VLCFAs as ALD disorders frequently have involvement with overt or subclinical adrenocortical insufficiency (Addison's Disease). Typically low DHEA levels have been identified in patient's not expressing clinical ALD symptoms (see, for example, Assies et al., 2003). Oral administration of hormones such as pregnenolone, DHEA or thyroid also stimulate peroxisomal proliferation via the β-oxidation of renegade fats as do nutrients (riboflavin, manganese), starvation states, the ketogenic diet or phospholipase A2 restrictive diet (reduced carbohydrate) and oxidative therapies (hyperbaric oxygen).

The limitation of aggressive stimulation of β-oxidation, however, is that not only are renegade fatty acids β-oxidized but essential fatty acids also are oxidized in the process and must be liberally replenished. Anti-oxidants are crucial nutrients but in excess they slow cellular metabolism and must remain in the proper balance with all the essential nutrients and substrates (e.g., essential fatty acids or EFAs) to maintain metabolic equilibrium. Inappropriate use (mega-dosing) of antioxidants such as Vitamin E will inhibit β-oxidation (Rudin, 1985) and the production of prostaglandins and cellular metabolism (Gurr, 2002). The very synthesis of a prostaglandin is an oxidative event and the liberal use of potent antioxidants would be contraindicated in the presence elevated VLCFAs, odd chain fatty acids and branched chain fatty acids in red cells, which may be indicative of toxicity (see, for example, Kane et al., 1996).

### Myelin, Neurolipids, and Large Brains

In 2005 Vargas (Vargus et al., 2005) and in 2006 Pardo (Pardo et al., 2006) described neuroinflammation with a unique proinflammatory profile of cytokines which is associated with increased oxidative stress in patients with autism. This phenomenon may lead to increased excitotoxicity. Minshew (Minshew et al., 1993) showed evidence of increased membrane degradation and decreased high-energy phosphate headgroups in the dorsolateral prefrontal cortex which relates to disturbances in cellular lipid structure, ceramide production, neuroinflammation and oxidative stress.

Enzymes involved in peroxisomal oxidation are suppressed by the elevation of inflammatory cytokines. Patients with autistic spectrum disorder often present with immune abnormalities as Pardo recently described (Pardo et al., supra) and may parallel with disturbances in peroxisomal function and impaired hepatic detoxification. Riboflavin (Vitamin B2) is pivotal in lipid metabolism, cytokine expression and exposure to endotoxins (Kodama et al., 2005), and may be used intravenously and/or orally to address inflammation, detoxification and peroxisomal function. Inflammation may involve the release of cytokines such as TNF-α (Tumor Necrosis Factor alpha) activating sphingomyelinases which in turn generate ceramides. Unlike the cytokines, the effects of increased sphingomyelin and ceramides are long lived and persist beyond the influence or the life of the inflammatory cytokines. Further, the metabolites of sphingomyelin inhibit sphingomyelin synthase and CTP:phosphocholine cytidylyltransferase (CT), inhibiting the normal ceramide-sphingomyelin homeostasis.

In 2002 Sokol (Sokol et al., 2002) found an increase in the choline/creatinine ratio associated with membrane degeneration. Upon examination of red cell fatty acids it has been found that in population of ASD and PDD patients 197 out of 300 subjects had increased levels of DMAs or Dimethyl acetyls (DMAs) or myelination biomarkers which may be indicative of increased or overmyelination. This is clearly a consistent pattern in patients with Amyothrophic Lateral Sclerosis (ALS) who have exceptionally elevated DMAs demonstrated in more than 400 individual's red cell lipid studies. (Kane, unpublished data).

Herbert has suggested (Herbert, 2003a, 2003b, 2005) that the ‘large brains’ in autism may be due to increased myelination. Bauman describes in the second edition of her book ‘The Neurobiology of Autism’ (Bauman and Kemper, 1^{st} edition, 1994 and 2^{nd} edition, 2005) that the most likely explanation for an increase in brain size is an abnormality in the formation of myelin which could lead to a disturbance in the processing of information throughout the brain. This phenomenon was described as a ‘possible quantitative difference’ in myelin phospholipids, proteolipid protein and glycolipids that could be aberrant in autism (Kohl, 2001) and (Greenfield et al., 2006). The finding of membrane enclosed brain glycolipids with adult presentation described by Simon (Simon et al., 1998) may support Kohl's hypothesis, especially as the siblings Simon describes present with symptoms that parallel autism.

An increased level of VLCFAs in patients with autism and neurological difficulties has consistently been observed (Kane et al., and Foster et al., supra) which may reflect deranged lipid metabolism in myelin as well as neuronal structures. Bauman and Kemper consistently found enlarged neurons (described as ‘big fat neurons’) in the brains (specifically in the deep cerebellar nuclei, inferior olive and nucleus of the diagonal bond of Broca in the septum) of children aged 5 to 13 years while in contrast older brains had neurons that were markedly reduced in size. (Bauman and Kemper, supra). Bauman presently hypothesizes that there may be various causation factors such as neuronal swelling which may be followed by atrophy due to transaction of an axon. Interestingly, in peroxisomal disorders the phenomenon of engorgement of very long chain fatty acids occurs in the initial phase but eventually is atrophied as the individual neurologically deteriorates or survives the metabolic disorder into adulthood (Kyllerman et al., 1990).

It has been postulated that fat brains or increased fat in the brain autopsies of stroke victims may consist of renegade or very long chain fatty acids that may have a relationship to impaired peroxisomal function as this has been noted in the red cell lipids of many stroke patients (Kane, International Conference Brain Uptake and Utilization of Fatty Acids, Bethesda Md. (2000) Unpublished results).

### Ceramides, Sphingomyelin and PC

Cellular membranes are comprised of bilipid layers of opposing phospholipids that line up soldier fashion and organize themselves spherically to provide the protective outer layer of every cell and the organelles within the cell. In the mammalian plasma membrane the two choline-containing phospholipids, phosphatidylcholine (PC) and sphingomyelin (SM), constitute more than 50% of the total phospholipid content of the membrane.

Ceramides typically contain saturated acyl chains ranging from 16 to 24 carbon atoms in length and are like a phospholipid with two fatty acid tails. The formation of ceramides encourage the formation of predominantly saturated fatty acids (FAs) on position 1 of the glycerol backbone, i.e. palmitic, and a very long chain (VLCFA) lignoceric or nervonic, both 24 carbon FAs, on the second position. The geometry of the membrane is highly sensitive to the size of the lipid chains. The width of the fatty acid portion of the membrane is approx. 3 to 4.5 nm including the head group, which must be maintained for stability. Saturated or monounsaturated FAs with a length of 16 or 18 carbons and polyunsaturated FAs of 18 to 22 carbons are preferred to permit the structure to maintain optimal horizontal fluidity. VLCFAs that range from 20 to 26 carbons force the parallel dimensions vertically or invade the opposing leaflet.

Ceramides lack a phosphate head group and are lipid molecules that combine with the choline head group from phosphatidylcholine (PC) to form sphingomyelin (SM). Originally thought to serve only as a structural function in membranes, SM is now recognized as serving complex signaling roles. Hijacking the choline head group to form SM from PC, SM, cholesterol as well as other low energy lipids e.g., additional ceramides, group together to form lipid rafts. The smaller head group of the ceramide, as well as the predominantly saturated fatty acids, encourages a tighter packing of the fatty acid chains in the membrane, which creates the formation of solid micro-domains (Mouritsen, 2005). Ceramides, however, are a prominent group of signaling molecules that arise from de novo SM synthesis and hydrolysis and are generated in response to oxidative stress and by receptor-mediated activation of sphingomyelinases. Mercury toxicity may participate in the apoptosis of nucleated cells, but only recently has been implicated in stimulating ceramide production (Eisele et al., 2006). Whereas cells under normal conditions contain very little ceramide, the ceramide content is increased up to about 10% of the lipid content upon apoptosis (Mouritsen, 2005). At low concentrations, sphingomyelin and ceramide can stimulate cell proliferation and survival, whereas higher levels can induce cell dysfunction or death.

Ceramides may play important roles in regulating processes such as cell proliferation, differentiation, and programmed cell death and have been implicated in the death of neurons that occur in ischemic stroke (Yu et al., 2000) and autism (Brugg et al., 1996). It has been reported that the effects of ceramide on the physical properties of the cell membrane are related to the molecular mechanisms behind apoptosis (Kinnunen et al., 2002). Ceramides can sensitize neurons to excitotoxic damage and thereby promote apoptosis. (Hofmann et al., 2000). There is evidence, however, linking the accumulation of ceramides and cholesterol esters with ROS (Reactive Oxygen Species) stress-induced death of motor neurons in amyotrophic lateral sclerosis (Cutler et al., 2002), neurons in Alzheimer's Disease (Cutler et al., 2004), in HIV-dementia (Haughey et al., 2004), stroke and in autism (Kane, unpublished data).

There is considerable evidence that links the production of ROS with ceramide generation and the subsequent loss of PC. SM formation increases with age, toxicity and disease with a consummate decline in PC (Cui and Houweling, 2002). These authors have reviewed the interaction between PC and cell death and discussed a variety of cellular disease states, both homeostatic and laboratory induced that perturb PC and lead to cell death. Alterations in PC homeostasis can occur during pathophysiological events (toxicity, infection) leading to aberrant PC homeostasis in mammalian cells and on to cell death. Cui and Houweling further stated that in a majority of studies of PC perturbation exogenous PC rescues cells from apoptosis.

### Essential Fatty Acids and the Specific Ratio of ω6 and ω3

The dry weight of the human brain, where enzymes which modulate lipids are strongly expressed, is about 60% lipid (Crawford et al., 1997), which in combination with dendrites and synapses comprises about 80% lipid (Peet et al., 1999). Phospholipids, cholesterol, cerebrosides, gangliosides and sulfatides are the lipids most predominant in the brain residing within the bilayers. The phospholipids and their essential fatty acid components provide second messengers and signal mediators and play a vital role in the cell signaling systems in the neuron (Rapoport, 1999). The functional behavior of neuronal membranes largely depends upon the ways in which individual phospholipids are aligned, interspersed with cholesterol, and associated with proteins. Neurotransmitters are wrapped up in phospholipid vesicles with the release and uptake of the neurotransmitters that are dependent upon the realignment of the phospholipid molecules. The nature of the phospholipid is a factor in determining how much of a neurotransmitter or metal ion will pass out of a vesicle or will be taken back in.

The optimal function of the membrane, and consequently the organism, is intimately dependent upon lipid substrates. The essential fatty acids must be ingested, and in a preferred proportion to one another, which involves the two basic essential fatty acid families (EFAs), ω6 and ω3 (omega 6 and omega 3). Without dietary or intravenous access to essential fatty acids and phospholipids the patient's condition is severely compromised.

Bourre and colleagues (Bourre et al., 1989) discovered that feeding rats a diet containing oils that were low in alpha linolenic acid (18:3 ω3) (ALA) content, such as corn or safflower oil, resulted in reduced amounts of docosahexaenoic acid (22:6 ω3) (DHA) in all brain cells and organelles compared to rats fed a diet containing soybean or canola oil. A diet low in ALA led to anomalies in the electroretinogram, with little effect on motor activity, but dramatically impacted learning. The dietary feeding of linoleic acid (LA) (18:2 ω6), however, had little effect on the level of DHA. The effect of ω3 on brain function from the work of Bourre and others stimulated similar essential fatty acid (EFA) research.

Of special importance was the work of Yehuda and colleagues, who in 1993 published their research on the discovery of optimized ratios of ω6 to ω3 and the benefits of the optimized ratio on the level of neuronal membrane function and neuronal transmission, expressed as the “membrane fluidity” index.

Cholesterol is a major membrane component, and along with the wax-like saturated palmitic and stearic acids, is responsible for the rigidity and strength of the membrane. EFAs such as polyunsaturated fatty acids (PUFAs) and highly unsaturated fatty acids (HUFAs) are liquid, or lipids that increase the fluidity index. An optimal index of high fluidity allows the exchange of ions between the inside and the outside of the membrane. This process is crucial for the transfer of neuronal information and for the proper activity of the ion channels.

The prior art research on EFA was conducted on small laboratory animals (rats) which possess a more efficient fatty acid metabolism than large mammals. Rodents are capable of metabolizing the base lipids LA and ALA up to HUFAs (GLA, DHGLA, AA, EPA, and DHA) since they are not burdened by the insufficiency of the rate limiting enzyme, delta 6 desaturase, as are large mammals, including humans. Incorporating the EFA ratios of the prior art requires consideration of the weaker human FA capability which necessitates the essential addition of dietary HUFA support such as meat, dairy, egg yolk and seafood, or fish oil supplements. The principal value of the higher ratio ω6 or ω3 FAs is the ability to raise the level of fluidity with a low risk of over-expression of either ω6 or ω3 FAs.

There is a long felt need for correct diagnosis, treatment and/or amelioration of the diseases that relate to the imbalance of essential fatty acids and cell membrane dysfunction, such as autism. The invention disclosed herein provides novel compositions and methods utilizing specific compositions and methods for diagnosis, treatment, or amelioration of the symptoms of such diseases and disorders. The invention disclosed herein evaluates the involvement of deranged peroxisomal lipid metabolism, compares other manifestations of lipid derangement in symptomatic or asymptomatic patients, and restore a healthy balance of essential nutrients in these patients, which are paramount to maintain or restore the health and thereby healing the symptoms of the disease.

### III. SUMMARY OF THE INVENTION

The invention as disclosed herein provides pharmaneutical compositions and methods for treating or ameliorating the symptoms of diseases associated with the imbalance of essential fatty acids.

In one aspect, the invention provides pharmaneutical compositions comprising an effective amount of a first and a second composition, the first composition comprises one or more phosphotidylcholine formulations and the second composition comprises one or more constituents comprising essential fatty acid supplements, trace minerals, butyrate (e.g., sodium phenylbutyrate), electrolytes, methylating agent, reduced glutathione, or a combination thereof, in a suitable carrier.

In one embodiment, the pharmaneutical composition further comprises peroxisomal cocktails including thiamin, riboflavin, pyridoxine, biotin, pantothenic acid, NADH, carnitine, CoQ10, or a combination thereof.

In another embodiment, the first composition, the second composition, or both are formulated in one or different solutions, and/or they are in the same or different formulations, such as, for example in a liquid or dry formulation.

In another embodiment, the first composition, the second composition, or both are administered contemporaneously or at different time intervals.

In yet another embodiment, the first composition, the second composition, or both are administered in a time-released manner.

In another embodiment, the essential fatty acid supplements comprise linoleic acid and alpha linolenic acid in a ratio of about 4:1.

In yet another embodiment, the methylating agents comprise vitamin B compounds, such as, vitamin B12 and B complex compounds. These compounds include, for example, methylcobalamin, folinic acid compounds comprising Leucovorin, Citrovorum, Wellcovorin, or a combination thereof.

In another embodiment, the trace minerals comprise E-Lyte Liquid Mineral™ set #1-8 containing separate solutions of biologically available potassium, zinc, magnesium, copper, chromium, manganese, molybdenum, and selenium.

In yet another embodiment, the electrolytes comprise sodium, potassium, chloride, calcium, magnesium, bicarbonate, phosphate, and sulfate, or a combination thereof, among others.

In another aspect, the invention provides a method of treating, ameliorating, or preventing the symptoms of the diseases and disorders related to an imbalance of essential fatty acids and cell membrane dysfunction in a subject, comprising administering to the subject an effective amount of a pharmanuetical composition comprising a first and a second composition, the first composition comprises one or more phosphatidylcholine formulations and the second composition comprises one or more constituents comprising essential fatty acid supplements, trace minerals, butyrate (e.g., sodium phenylbutyrate), electrolytes, methylating agent, reduced glutathione, or a combination thereof, in a suitable carrier or diluent, wherein the symptoms of autism in the subject are treated, ameliorated, or prevented.

In one embodiment the disease or disorder is autism.

In another embodiment, the pharmaneutical composition further comprises peroxisomal cocktails containing thiamin, riboflavin, pyridoxine, biotin, pantothenic acid, NADH, carnitine, CoQ10, fatty alcohols (e.g., VIOBIN®, PROMETOL®), or a combination thereof and the subject is on a nutrient dense PLA2 suppressive diet.

In yet another embodiment, the first composition, the second composition, or both is administered intravenously, orally, or both.

In another embodiment, about 250 mg to 500 mg phosphatidylcholine is administered to the subject intravenously by lipid exchange once to three times daily for about two to four days a week, and bolus amounts of phosphatidylcholine are used intravenously by IV drip as 2 grams to 5 grams at least once weekly (e.g., once, twice, three times or more weekly) or at least once monthly (e.g., once, twice, three times, four times or more monthly). About 3600 mg to about 18,000 mg of phosphatidylcholine is administered to the subject daily by mouth.

In another embodiment, sodium phenylbutyrate is administered intravenously by IV drip as 1 gram to 4 grams once monthly to once weekly.

In another embodiment, about 910 mg to about 2600 mg of gamma linolenic acid contained in evening primrose oil is administered to the subject daily by mouth.

In yet another embodiment, about 30 mls to about 60 mls of the essential fatty acids (EFAs) 4:1 is administered to the subject daily by mouth.

In another embodiment, trace minerals are administered to the subject up to three times daily.

In another embodiment, oral electrolytes are administered to the subject up to five times daily.

In another embodiment, methylating agents such as folinic acid as Leucovorin is administered to the subject intravenously as 2 mg (0.2 cc) to 5 mg (0.5 cc) twice to three times daily for about three to four days a week in addition to twice weekly injections of 2 mg to 5 mg of methylcobalamin.

In yet another embodiment, reduced glutathione is administered intravenously at about 1800 mg to about 2400 mg, 1-3 times daily, and for 2-4 days in a seven-day period and the subject is maintained on a low carbohydrate, high protein, and high fat diet.

In yet another embodiment, the invention provides a method of treating, ameliorating, or preventing the symptoms of autism in a subject, comprising:
i) intravenous administration of a phosphatidylcholine composition comprising about 250 mg to 500 mg phosphatidylcholine followed by intravenous administration of Leucovorin (Folinic Acid) as 2 mg (0.2 cc) to 5 mg (0.5 cc), and followed by intravenous administration of about 200 mg to about 1200 mg of reduced glutathione, once, twice of three times daily for about 3 to 5 days in a seven-day period; ii) once daily oral administration of a phosphatidylcholine composition comprising about 3600 to about 18,000 mg of phosphatidylcholine daily; iii) once or twice daily oral administration of an effective amount of one or more trace minerals; iv) once daily oral administration of about 30 mls to about 60 mls of an EFA 4:1 composition; v) once daily oral administration of about 910 mg to about 2600 mg of gamma linolenic acid in evening primrose oil; vi) oral administration of 1 oz oral electrolytes are administered up to five times daily and vii) once daily oral sublingual or injectable administration of about 0.2 cc (2 mg) to about 0.5 cc (5 mg) two times weekly of Methylcobalamin, wherein the subject is treated or the symptoms of autism in the subject is treated, ameliorated, or prevented.

In yet another aspect, the invention provides a kit for the treatment, amelioration, or prevention of the diseases related to imbalance of essential fatty acids and cell membrane dysfunction in a subject, comprising: a) a first composition comprising one or more phosphatidylcholine formulations; b) a second composition comprising one or more constituents comprising: i) essential fatty acid supplements; ii) trace minerals; iii) butyrate or phenylbutyrate; iv) electrolytes; v) methylating agents folinic acid as Leucovorin and methylcobalamin; and vi) glutathione, c) instructions for the use of the first and second compositions; and d) instructions for where to obtain any missing components of the kit. The kit can further comprise instructions for determining an effective amount of the trace minerals for administration to the subject.

In one embodiment, the first composition, the second composition, or both are formulated in one or different solutions.

In another embodiment, the methods and compositions of the invention are used in combination with other commonly used treatments, and/or medications for treatment of autistic disease and disorders.

Other preferred embodiments of the invention will be apparent to one of ordinary skill in the art in light of what is known in the art, in light of the following description of the invention, and in light of the claims.

### III. BRIEF DESCRIPTION OF THE DRAWINGS

FIG. 1. Bargraph demonstrating fatty acids distribution in RBC lipids in children with ASD and PDD. The concentration of VLCFA, DHA, odd chain fatty acids, EPA, DMA, branched chain fatty acids, total lipid content and total omega 6 fatty acids were measured in the RBC of autistic children. Low, normal and high values of each of the fatty acids are indicated in the barograph.

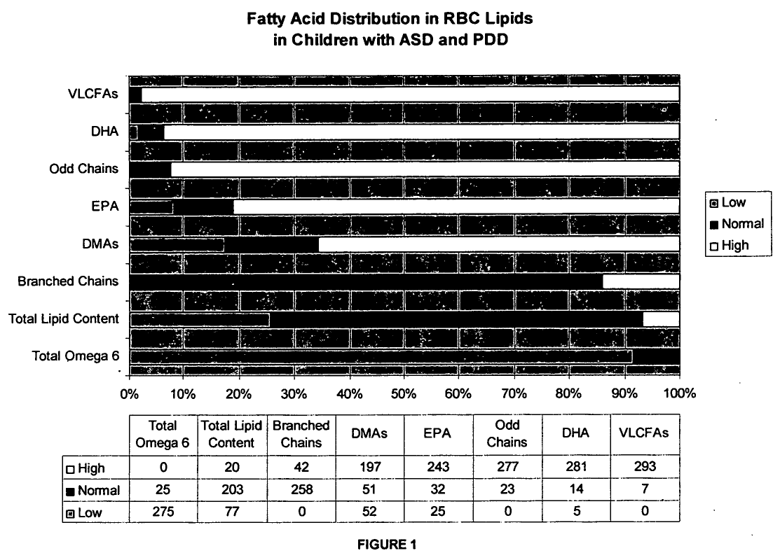

FIG. 2. Bargraph demonstrating individual renegade fatty acid distribution of RBC lipids in children with ASD and PDD. The percentage of VLCFA, DHA, odd chain fatty acids, EPA, DMA, branched chain fatty acids, total lipid content and total omega 6 fatty acids were measured in the RBC of autistic children. The high value for each of the fatty acids is indicated in the bargragh.

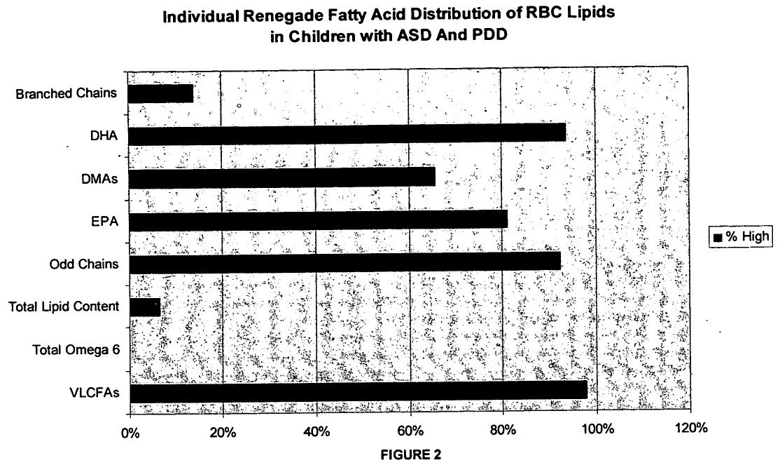

### IV. DETAILED DESCRIPTION OF THE INVENTION

The invention as described herein provides pharmaneutical compositions and methods for treating amelioration and/or prevention of diseases and disorders related to cell membrane dysfunction and imbalance and derangement of fatty acids indicative of cell membrane instability.

The methods and compositions of the invention treat, prevent and/or ameliorate a wide spectrum of diseases and disorders that are caused by or results from cell membrane dysfunction and imbalance and derangement of fatty acids. The diseases and disorders include, by way of example and not limitation, autism, pervasive developmental delay, seizure disorders, epilepsy, cerebral palsy, premature birth, infertility, brain injury with or without oxygen deprivation, methylation defects, polymorphism, psychosis, bipolar, schizophrenia, mood disorders (e.g., depression, anxiety, ADD, and ADHD), ALS, Parkinson's Disease, multiple sclerosis, Alzheimer's Disease, Huntington's Disease, drug addiction, alcoholism, environmental illness, cardiovascular disease, stroke, hypercholesterolemia, hypertriglyceridemia, respiratory disease, hepatic disease, kidney disease, macular degeneration, skin disorders such as gross eczema, Hepatitis C, Lyme disease, Fibromyalgia, chronic fatigue syndrome, hepatic encephalopathy, meningitis, encephalitis, systemic sepsis, and toxic exposure to pesticides, chemicals, solvents, heavy metals, and microbials such as mycotoxins (mold, fungus), bacteria, virus, mycoplasma, trigeminal neuralgia, among others.

The symptoms of diseases and disorders related to essential fatty acid imbalance and cell membrane dysfunction include, by way of example and not limitation, elevation of very long chain fatty acids (renegade fatty acids) and derangement of fatty acids indicative of cell membrane instability, elevation of DHA (Docosahexaenoic acid), elevation of myelination markers (e.g., DMA (dimethyl acetyls)), suppression of essential fatty acids, low cholesterol, increase in blood urea nitrogen (BUN), electrolyte disturbance, decrease in IGF1 (insulin growth factor 1), decrease in hormones (e.g., DHEA, pregnenolone, alpha MSH), polymorphisms of methylene tetrahydrofolate reductase (MTHFR) (as A1298C 1 or 2 copies, and C677T (1 or 2 copies), elevation of liver enzymes (e.g., GGT, LDH, SGOT, SGPT), increase of RDW (radius of the red cell) and uric acid, both indicative of poor methylation, elevation of creatine kinase (depicts low PC), elevation of potassium in blood chemistry indicative of poor cell membrane integrity, disturbance in urinary neurotransmitters, especially elevation of glutamate, and aspartic acid with suppression of serotonin and GABA, and disturbance in urinary organic and amino acids, among others.

In particular, the invention provides compositions and methods for treating, ameliorating and/or preventing the symptoms of autism and inhibiting the progression of the disease using a composition containing nutritional and natural supplements as disclosed herein.

As used herein, “autism”, and Autistic Spectrum Disorder (ASD) are used interchangeably herein. Both autism and ASD may include one or more symptoms of pervasive developmental delay (PDD), among other biological, social and psychological symptoms.

As used herein, a “pharmaneutical composition” includes any composition in which at least 50% of its compounds, compositions and/or constituents have been derived from natural sources and/or are used in their natural form, as opposed to being chemically, or synthetically produced.

As used herein, a “subject” is any mammal, in particular a primate, preferably a human, that 1) exhibits at least one symptom associated with autism; 2) has been diagnosed with autism; or 3) is at risk for developing autism.

As used herein, a “subject at risk for developing autism” includes subjects with a family history of autism or who are susceptible to developing autism. Subjects “susceptible to developing autism” include those subjects testing positive for molecular markers indicative of or associated with autism, or demonstrate behavioral or psychological patterns indicative of autism. However, some patients can find that getting a diagnosis of autism is a challenge. There are no medical diagnostic tests for autism, meaning that a brain scan does not diagnose it. CT scan or MRI of the brain is not able to show most microscopic changes that happen in the brain, and most patients with autism will have normal brain scans.

As used herein, an “effective amount” of a composition is an amount sufficient to achieve a desired biological effect, in this case at least one of prevention, amelioration or treatment of autism. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

As used herein, a “carrier” refers to a diluent, adjuvant, excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Sterile water is a preferred carrier when the pharmanuetical composition is administered intravenously. Saline solutions and aqueous dextrose and glycerol solutions can also be employed as liquid carriers, particularly for injectable solutions.

As used herein, Glutathione, and rglutathione (Reduced Glutathione) are used interchangeably herein.

The systemic nature of Autistic Spectrum Disorder (ASD) and the pervasive developmental delay (PDD) that may accompany it has led the inventor of the invention to view the complexity of these presentations by addressing them from a cell membrane perspective. Autistic spectrum disorder is a complex neurodevelopmental disorder, which has been investigated under an open clinical study rather than randomized, placebo, controlled and double blinded studies due to the constellation of symptoms in autistic spectrum disorder and multiple variables in regard to the oral and IV intervention. Each subject served as his or her own control for several reasons, 1) control pediatric subjects are difficult to obtain due to the use of intravenous therapy, 2) compliance in regard to a restricted carbohydrate diet and oral intake of supplements is limited in controls, and 3) matching two subjects with the same diagnosis, gender, age, development, metabolism and presentation is unrealistic in regard to autism and PDD. Presently there are no biological markers that exist to identify autistic spectrum disorder in addition to speculation that multiple, variable susceptibility genes, epigenetic effects, and environmental factors complicate the disorder we term autism.

Examination of red cell lipids in subjects with ASD and PDD over the past decade in more than 7000 analyses has revealed an accumulation of very long chain fatty acids (VLCFAs) in red cells, which are components of ceramides and lipid rafts indicative of cell membrane derangement. Membrane phospholipid abnormalities with elevation of VLCFAs are suggestive of exposure to neurotoxins resulting in reduced expression of peroxisomal β-oxidation. Disturbances in methylation due to toxic exposure destabilize the membrane phospholipid structure and alter DNA expression due to deficits in enzymes such as, for example, Methylene Tetrahydrofolate Reductase (MTHFR) and Methionine Synthase.

According to one embodiment of the invention, there is provided a clinical treatment plan to clear the bioaccumulation of toxins and stabilize membrane function. The method of treatment according to this embodiment of the invention, addresses the accumulation of aberrant lipids and toxins with oral and intravenous phospholipids (phosphatidylcholine as LipoStabil™ or Essentiale N™), balanced ω6 and ω3 fatty acids, methylation factors (Leucovorin, folinic acid, riboflavin tetrahydrobiopterin, and methylcobalamin), butyrate or sodium phenyl butyrate, and intravenous reduced glutathione. The use of oral and IV lipids facilitates stabilization of phospholipids in cellular membranes thereby addressing hepatic and CNS clearance of microbes, chemicals and heavy metals. Heavy metals and microbes are fat soluble and therefore cellular soluble rendering chelating agents and antibiotics limited in hepatic and CNS tissues.

A dramatic and sustained clinical improvement has been observed within the first few weeks after initiation of oral and intravenous treatment of the invention in the patient population of over 300 subjects with autistic spectrum disorder. A review of the collective laboratory data of red cell fatty acids of these subjects was performed in order to evaluate the possible involvement of deranged peroxisomal lipid metabolism and to compare other manifestations of lipid derangement in children with autism. Additionally, the complex integration of disturbed lipid metabolism which is involved in alterations of detoxification and CNS function was observed.

The invention disclosed herein provides IV and oral treatment protocols that address clearance of possible neurotoxins, yield stabilization of membrane phospholipids and balance the essential fatty acids. A few of the individual case studies are presented for clarification of the diversity of the autistic spectrum presentation and detail on individual response to the inventive targeted IV and oral therapy.

In another embodiment, the invention provides a method of treating or reversing prevalent symptoms of autism in pediatric patients with autistic spectrum disorder and especially in autistic patients with disturbed lipid metabolism and impaired detoxification by administration of a phospholipid therapy with glutathione and methylation and sodium phenylbutyrate.

The pharmaneutical compositions and methods of the invention are designed on the principle of “balanced nutrients” and “stabilization of phospholipids within the cell membrane”. The normal body keeps a healthy balance among essential nutrients that is a key in the well being and health of the individual. Unlike most therapies that cause an imbalance in the body of a sick individual who is already comprised by the sickness or the disease itself, therapeutic methods of the present invention heal the subject individual by restoring the balance of essential nutrients to adjust it to a normal level in order to assist the body to fight the abnormal condition and/or ailments and to increase the ability of the immune system to fight the disease.

In states of toxicity via biotoxins or heavy metals there is a sharp elevation in Phospholipase A₂ (PLA₂) activity. Increases in PLA2 activity result in premature uncoupling of the essential fatty acids (EFAs) from phospholipids in the cell membrane. Accelerated loss of EFAs places the patient in a severely compromised position as that of inflammation, which results from the promiscuous release of arachidonic acid (AA) in the presence of an overexpression of PLA₂.

There are more than 19 different isoforms of PLA₂ that have been identified, but there are three major PLA₂s that are focused upon as 1) secretory PLA₂ (s PLA₂) which is secreted by the pancreas, neurons, inflammatory cells and damaged tissues in addition to 2) intracellular calcium (2+)-independent PLA₂ (i PLA₂) and 3) cytosolic PLA₂ (c PLA₂). PLA₂s are enzymes defined by their ability to catalyze the hydrolysis of the middle (sn-2 position) ester bond of phospholipids. PLA₂s are involved in signaling pathways that link receptor agonists, oxidative agents, and proinflammatory cytokines to the release of arachidonic acid (AA) and the biosynthesis of eicosanoids. At low concentrations PLA₂s act on membrane phospholipids and are involved with intracellular membrane trafficking, proliferation, differentiation, and apoptotic processes. At high concentrations, however, PLA₂s are cytotoxic. Severe neurodegeneration may occur in the brain if PLA₂ activity is not controlled. The elevation of cytosolic phospholipase A₂ is reported to be linked to psychiatric conditions known as Phospholipid Spectrum Disorder.

Mercury is known to be one of the most potent stimulators of PLA₂ Elevation of TNF-α is also known to be a major contributor to the release of PLA₂ and destabilization of the membrane lipids. Glucose-induced insulin secretion via high consumption of refined carbohydrates is a strong stimulator of PLA₂ and must be restricted to control the wasting of EFAs released from the phospholipids. Of further concern is that excessive carbohydrate consumption, as is the case with many diets of children with ASD, may lead to periods of hyperinsulinism which may inhibit hepatic peroxisomal beta-oxidation.

It has been found that both PKC-α, protein kinase (MAPK), and cytosolic phospholipase A₂ (cPLA₂) are required for the ceramide-induced inhibition of Phosphocholine cytidylyltransferase (CT) activity. Based on this data and findings in the literature, it is also suggested that the inhibition of CT is from the generation of lysoPC through the action of activated cPLA₂. Arachidonic acid, the direct product of cPLA₂ hydrolysis, is a substrate for prostaglandins (PGE2) and leukotrienes, which may stimulate Ca²⁺ influx and thereby further activate cPLA₂. The ultimate loss of PC is therefore a downstream effect of inflammation from over stimulated cPLA₂, by increasing lysoPC and AA as well as Ca²⁺ influx. Potent inhibitors of PLA₂, in states of overexpression, include lithium, intravenous glutathione, phosphatidylcholine and limited carbohydrate consumption.

Patients with ASD and PDD often have both a heavy metal burden co-existing with the additional complication of the presence of biotoxins. Heavy metals are lipid soluble and often compound the removal of biotoxins. A variety of toxins may co-exist within the cell membrane and fatty tissue requiring consideration for a variety of toxins (pesticides, petrochemicals, neurotoxic mold, bacteria, virus, parasites, heavy metals, chemicals such as acetaminophen) thus intervention must address all aspects of possible toxins involved in the presentation. Adding more medication often further damages a system that is already compromised.

The introduction of a phospholipid emulsion as phosphatidylcholine, LipoStabil™, in accordance with one embodiment of the invention, clears lipid soluble microbes and toxins from the body. Initially, research was conducted on animals whereby meningitis and systemic sepsis were cleared by the use of intravenous bolus phosphatidylcholine. Human trials were later conducted on the use of IV bolus phosphatidylcholine to establish the safety of doses of 7 grams, 14 grams and 21 grams in which no side effects were observed.

The treatment methods of the invention has application in treating diseases resulting from neurotoxic mold, heavy metal burdens, chronic lyme disease, pesticide poisoning, and chronic viral syndromes. The primary focus with the use of our intravenous PC-Leucovorin-GSH clinical procedure, however, has been with adult and pediatric patients with neurological involvement.

In one embodiment, the method of the invention provides an intravenous administration with phospholipid exchange or bolus phospholipid drip followed by IV Leucovorin (folinic acid) to support methylation. In the last step of this protocol, the reduced glutathione (diluted with sterile H₂0). One objective for administration of Glutathione with PC is to achieve a lipid soluble glutathione via micelle delivery to chelate heavy metals bound to metallothionein. Other actions of glutathione comprise supporting immune function, suppressing PLA₂ and thereby stabilizing the phospholipids in the membrane, inhibiting TNF-α, and act in an anti-inflammatory capacity. Glutathione acts as a versatile and pervasive metal binding ligand and forms metal complexes via nonenzymatic reactions. The sulfhydryl group of the cysteine moiety of glutathione has a strong affinity for mercury, silver, cadmium, arsenic, lead, gold, zinc, and copper. Glutathione acts in the transport of the metal across cell membranes, works in the mobilization and delivery of metals between ligands and perform as a reductant or cofactor in redox reactions involving metals, among other actions.

In another embodiment, the invention provides a method of treating autism with both oral and intravenous lipid therapy. One objective of the methods of the invention is to attenuate the accumulation of ceramides and renegade fatty acids which can compromise hepatic and CNS function. Oral and IV Phospholipid and Phenylbutyrate therapy modifies the neuronal and hepatic membrane distortion by displacing the subsequent early expression of sphingomyelin which follows the rise of ceramide synthesis.

In one embodiment, phosphatidylcholine is administered first so that the glutathione may become lipid or cell soluble. By stabilizing the patient with intravenous glutathione, the method of the invention impacts metallothionein markers, glycoaminoglycans or GAGS, methylation, sulfation, hepatic and renal function. The method of the invention introduces treatment protocols for detoxification with a gentle, natural modality that unloads cellular toxicity safely. The intravenous PC-Leucovorin-GSH protocol of the invention has clinically demonstrated to be supportive in the release of the body burden of heavy metals and toxins in both pediatric and adult populations and without any side effects that are normally associated with the use of chemical chelators. The inventive bolus dosing with PC as an intravenous drip followed by two infusions of Leucovorin and GSH has yielded significant urinary spills of toxic metals including arsenic, lead, cadmium, mercury and antimony. Repeated dosing with the PC lipid exchange or bolus method followed by Leucovorin-Glutathione of, for example, one, two or more infusions daily for about 3, 4, 5, 6, or 7 days, or once, twice or three times on a weekly interval has also resulted in significant toxic metal release in the urine.

The primary source of heavy metal exposure for many patients has been in fetal development with the mother's exposure to high daily amounts of fish, most commonly white albacore tuna being consumed daily. In one case, the mother of a patient with severe autism reported that she ate swordfish every day of her pregnancy. Methylmercury exposure is almost always exclusively dietary and although there are many environmental exposures to various forms of mercury, it is methylmercury which can be the most devastating to the CNS and brain. Chelation with chemical agents such as meso-2,3-dimercaptosuccinic acid (DMSA) does not impact the body burden of toxic metals in regard to the brain and CNS functions even though DMSA was an effective agent at removing lead.

Baseline testing for toxic metal exposure is problematic. The elusiveness of collecting evidence of chronic mercury exposure or most neurotoxins for that matter is perplexing to the clinician and to researchers alike. The invasive methods of brain or liver biopsy and lack of accuracy of these methods and other laboratory analysis make these methods undesirable for most patients. Often, the physician must rely on the aftermath of exposure to a potential neurotoxin as the patient presents upon history, physical and examination along with biochemical alterations observed in blood chemistry with complete blood count, and red cell fatty acids.

The cellular impact of toxins and heavy metal burdens results in disturbed prostaglandin synthesis, poor cellular integrity, increased cytokines, decreased GSH levels, significant suppression of ω6 arachidonic acid and marked elevation of renegade fats and ultimately with disturbed myelination, among other symptoms. Suppression of inflammatory cytokines [TNF-α, IL-1β (Interleukin-1β), interleukin (IL)-6, IL-10, superoxide dismutase (SOD) and malondialdehyde (MDA)], protection from lipid peroxidation, reduction of total nitrite/nitrate (NOx), and hepatic and cytoprotective effects have been demonstrated with the use of phosphatidylcholine.

Damage may occur to the blood brain barrier with elevation of cytokines such as TNF-α. As glutathione may suppress TNF-α, in one embodiment of the invention, it is administered intravenously with PC to maximize its potential of entry through the cell membrane and BBB. The concept of the IV use of PC is that of rejuvenating membranes and cells and an attempt to promote a consummate increase in fluidity due to the high concentration of essential fatty acids with a multitude of cis-double bonds within the PC. The treatment methods of the invention address cellular derangement by introducing PC, both orally and by IV infusion, to potentially offset the accumulation of ceramides, influence fluidity, clear neurotoxins, and stabilize the integrity of the lipid membrane leaflets.

1. Description of Pharmaceutical Constituents

1.1 Phosphatidylcholine

Phosphatidylcholine (PC) is the predominant phospholipid of all cell membranes and of the circulating blood lipoproteins. PC is the main lipid constituent of the lipoprotein particles circulating in the blood and the preferred precursor for certain phospholipids and other biologically important molecules. PC also provides antioxidant protection in vivo. In animal and human studies, PC protected against a variety of chemical toxins and pharmaceutical adverse effects.

Chemically, PC is a glycerophospholipid that is built on glycerol (CH2OH—CHOH—CH2OH) and substituted at all three carbons. Carbons I and 2 are substituted by fatty acids and carbon 3 by phosphorylcholine. Simplistically, the PC molecule consists of a head-group (phosphorylcholine), a middle piece (glycerol), and two tails (the fatty acids, which vary). Variations in the fatty acids in the tails account for the great variety of PC molecular species in human tissues.

In vivo, PC is produced via two major pathways. In the predominant pathway, two fatty acids (acyl “tails”) are added to glycerol phosphate (the “middle piece”), to generate phosphatidic acid (PA) that is converted to diacylglycerol, after which phosphocholine (the “head-group”) is added on from CDP-choline. The second, minor pathway is phosphatidylethanolamine (PE) methylation, in which the phospholipid PE has three methyl groups added to its ethanolamine head-group, thereby converting it into PC.

Taken orally PC is very well absorbed, up to 90% per 24 hrs when taken with meals. PC enters the blood gradually and its levels peak over 8-12 hours. During the digestive process, the position-2 fatty acid becomes detached (de-acylation) in the majority of the PC molecules. The resulting lyso-PC readily enters intestinal lining cells, and is subsequently re-acylated at this position. The position-2 fatty acid contributes to membrane fluidity (along with position I), but is preferentially available for eicosanoid generation and signal transduction. The omega-6/omega-3 (ω6 or ω3) balance of the PC fatty acids is subject to adjustment via dietary fatty acid intake. Choline is most likely an essential nutrient for humans, and dietary choline is ingested predominantly as PC. Greater than 98 percent of blood and tissue choline is sequestered in PC that serves as a “slow-release” blood choline source.

Methyl group (—CH3) availability is crucial for protein and nucleic acid synthesis and regulation, phase-two hepatic detoxification, and numerous other biochemical processes involving methyl donation. Methyl deficiency induced by restricted choline intake is linked to liver steatosis in humans, and to increased cancer risk in many mammals. PC is an excellent source of methyl groups, supplying up to three per PC molecule, and is the main structural support of cell membranes, the dynamic molecular sheets on which most life processes occur. Comprising 40 percent of total membrane phospholipids, PC's presence is important for homeostatic regulation of membrane fluidity. PC molecules of the outermost cell membrane deliver fatty acids on demand for prostaglandin/eicosanoid cellular messenger functions, and support signal transduction from the cell's exterior to its interior.

PC compositions used within the scope of the invention include, by way of example and not limitation, compositions comprising phosphatidylcholine including Essential N™ or LipoStabil™ 500 mg to 1000 mg phosphatidylcholine used intravenously by lipid exchange or in a bolus IV solution as 2 grams to 5 grams, available from BodyBio Inc. (Millville, N.J. USA).

1.2 Essential Fatty Acids (EFAs)

Essential Fatty Acids (EFAs) are long-chain polyunsaturated fatty acids derived from linolenic, linoleic, and oleic acids. EFAs are necessary fats that humans cannot synthesize, and must be obtained through diet. EFAs compete with undesirable fats (e.g. trans fats and cholesterol) for metabolism. Also, EFAs raise the HDL (High Density Lipoprotein) that is also considered beneficial for the body by capturing the undesirable LDL (Low Density Lipoprotein), and escort it to the liver where it is broken down and excreted.

There are two families of EFAs: Omega-3 and Omega-6. Omega-9 is necessary yet “non-essential” because the body can manufacture it in a modest amount, provided essential EFAs are present. The number following “Omega-” represents the position of the first double bond, counting from the terminal methyl group on the molecule. Omega-3 fatty acids are derived from Linolenic Acid, Omega-6 from Linoleic Acid, and Omega-9 from Oleic Acid.

EFAs support the cardiovascular, reproductive, immune, and nervous systems. The human body needs EFAs to manufacture and repair cell membranes, enabling the cells to obtain optimum nutrition and expel harmful waste products. A primary function of EFAs is the production of prostaglandins, which regulate body functions such as heart rate, blood pressure, blood clotting, fertility, conception, and play a role in immune function by regulating inflammation and encouraging the body to fight infection. Essential Fatty Acids are also needed for proper growth in children, particularly for neural development and maturation of sensory systems, with male children having higher needs than females. Fetuses and breast-fed infants also require an adequate supply of EFAs through the mother's dietary intake. Because high heat destroys linolenic acid, cooking in linolenic-rich oils or eating cooked linolenic-rich fish is unlikely to provide a sufficient amount.

EFA deficiency is common in the United States, particularly Omega-3 deficiency and now Omega-6 deficiency due to the increased use of hydrogenated vegetable oil, and recently, over prescribing and consumption of Fish Oil. Essential fatty acid supplements include solutions comprising a mixture of omega 6 and omega 3 fatty acids, in ratio of from about 20:1, 10:1, 5:1, 4:1, 3:1, 2:1, 1:1, or less. It is intended herein that by recitation of such specified ranges, the ranges recited also include all those specific integer amounts between the recited ranges. For example, in the range of about 4:1, it is intended to also encompass 4.2:1, 3.8:1, 3.5:1, 3.2:1, 3:1, etc, without actually reciting each specific range therewith. Preferably the ratio between the omega 6 and omega 3 fatty acids is about 4:1 v/v.

Incorporating the 4:1 ratio requires consideration of the weaker human FA (fatty acids) capability which necessitates the essential addition of dietary HUFA (highly unsaturated fatty acids) support such as meat, dairy, egg yolk, seafood, or fish oil supplements. The principal value of the 4:1 ratio is the ability to raise the level of fluidity with a low risk of over-expression of either ω6 or ω3 FAs. Clinical application of EFA 4:1 gives the clinician a critically important tool to raise EFAs and subsequently fluidity to a higher level and maintain that critical balance. Balancing EFAs with about 80% ω6s will in effect contribute to the formation of Arachidonic acid (AA).

AA (20:4ω6) is a 20 carbon HUFA with 4 double bonds and is the lead eicosanoid for the production of prostaglandins, thromboxanes and leucotrienes. Arachidonic acid (AA) is a prominent essential fatty acid in red blood cells as 15% and total brain lipids are comprised of 12% AA. All fluidity comes from the double bonds (DB) of the MUFA (monounsaturated fatty acids), PUFA (polyunsaturated fatty acids), and HUFA (highly unsaturated fatty acids) with the most prominent coming from the ω6s. A review of the melting point of each lipid helps to visualize the contribution of the DBs. Palmitic and stearic, both saturated have a melting point of about 65° C. and it accounts for about 32% of the red cell membrane. Since the body has a temperature of 37.5° C., palmitic acid (PA) and stearic acid (SA) are solid in the membrane of animals. Oleic acid (OA), a monounsaturated FA with one DB is liquid at 16° C., it accounts for about 10.2% of red cell fatty acids and is the beginning of fluidity.

TABLE 1
EFAs, double bonds, fluidity contribution, melting point.

| | Double | % of Red | Total DBs | Melting | |
|---|---|---|---|---|---|
| | Bonds | Blood Cells | (Dbs × %) | Point | |
| The ω6s: | | | | | |
| Linoleic (LA) | 2 DB | 10.28% | 20.56 | −5° | C. |
| Gamma Linolenic (GLA) | 3 DB | 0.07% | 0.21 | −11° | C. |
| Dihomogamma Linolenic | 3 DB | 1.47% | 4.41 | −11° | C. |
| Arachidonic (AA) | 4 DB | 15.07% | 60.28 | −49° | C. |
| Adrenic | 4 DB | 3.48% | 13.94 | | |
| The ω3s: | | | | | |
| Alpha Linolenic | 3 DB | 0.28% | 0.9 | −11° | C. |
| Eicosapentaenoic | 5 DB | 0.44% | 2.2 | −55° | C. |
| Docosapentaenoic | 5 DB | 2.06% | 10.3 | | |
| Docosahexaenoic | 6 DB | 3.46% | 20.76 | −59° | C. |

Multiplying the DBs times the percent fatty acid concentration; the total value for the ω6s is 99.40 compared to the ω3s at 34.16. Clearly the ω6s are the prominent FA in the human body with close to 3 times the energy value of the ω3 s. The numbers reverse themselves with the ω3s taking prominence in the brain with the much higher concentration of DHA at about 17-22% and especially in the outer segments of the photo receptor cells in the retina at about 55%. Viewing the double bonds as a storehouse of energy presents a different picture of AA than currently held in the popular literature. The disturbing picture of AA is grossly misrepresented as is its metabolic value. Lacking sufficient arguments for any suppression of arachidonic acid as well as the suppression of any other FAs, we have found that the proper balance of the amino acids must be made on a case by case basis on the basis of the individualized biochemical data, such as for example, individual's red cell lipid analysis. However, the promiscuous use of marine oil, as is the case in a surprising number of patients, has resulted in gross distortion of their red cell fatty acids.

Elevation of DHA (Docosahexaenoic acid) in the red cell lipids of children with autism was first reported by Kane. An increase in Docosahexanenoic acid or DHA in the analysis of red cell fatty acids is indicative of neuroinflammation, the increased release of nitric oxide and aberrant lipid metabolism following toxic exposure. Patients with autism and other neurological disorders such as seizures, ALS, Multiple Sclerosis, Parkinson's and Alzheimer's disease often have elevation of DHA with or without supplemental fish oil or the consumption of fish in the diet. The elevation of DHA is a biomarker of neuroinflammation due to aberrant lipid metabolism after toxic exposure which may result in abnormal lipid metabolites of DHA being formed. Kane and Kane U.S. co-pending patent applications, application Ser. Nos. 11/171,308, and 10,946, 601, each of which is incorporated herein by reference in its entirety.

Over the past 10 years the phenomenon of an omega 3 overdose syndrome has been prevalent. More common symptoms in pediatric patients are hypotonia and lethargy (if high EPA formulas were used), eczema or other skin eruptions, inflammation, lack of speech, poor responsiveness, learning difficulties, irritability, and seizures. Pediatric patients appear to have significant re-stabilization of arachidonic acid, not GLA and DGLA, with aggressive oral balanced HUFA lipid therapy (egg yolk, meat fat, evening primrose oil) within about 6 months from the time that marine oil has been overdosed.

The phospholipid therapy of the invention expedites stabilization of balanced phospholipids in the membrane in both our adult and pediatric populations. In one embodiment, the treatment is via IV administration of a phosphatidylcholine derived from soy composed of 50% dilinoleoylphosphatidylcholine.

The use of excessive quantities of marine or flax oil can suppress the ω6s, reflected in the lower concentration of AA (arachidonic acid) which can disturb the balance of eicosanoids. As research emerges on the complexity of the interaction of the higher order ω6 to ω3, it is becoming more evident that balance of ω6 to ω3 is paramount. It has been found that when EPA was supplemented but not other long-chain n-3 or n-6 PUFA there was a decrease natural killer cell activity in healthy subjects. When arachidonic acid is suppressed due to excess intake of omega 3, toxicity or disease, the body is perturbed which is clearly viewed in the patient's red cell fatty acid analysis. Arachidonic acid is preferentially wasted in states of heavy metal toxicity and has been observed to be sharply suppressed in red cell fatty acid analysis in states of heavy metal toxicity (Kane et al., 2002a). Arachidonic acid is reduced in serum concentrations in pregnant women and their infant's cord blood with exposure to polychlorinated biphenyls (PCBs) indicative of desaturase inhibition.

Heavy metals and biotoxins can be recycled via bile and the patient can be repeatedly exposed to these toxins through enterohepatic circulation. The presence in the red cells of high VLCFAs is suggestive of peroxisomal dysfunction, suppression of the beta oxidation of lipids and cellular respiration, which may be exacerbated by exposure to heavy metals. Biotoxins and heavy metals are lipid soluble thus the effect upon cellular processes and hepatobiliary function is often aberrant. Alteration of peroxisomal fatty acid metabolism leads to reduced expression of peroxisome proliferator-activated receptor-alpha (PPAR-α) possibly leading to the development of the fatty liver disease. The consumption of fats and oils is often avoided, but if taken it improperly digested if the gall bladder is not functioning properly. Cholestasis or steatosis is often present which may inhibit the release of glutathione from the liver. PPARs (alpha, gamma, delta) are HUFA lipid-activated nuclear transcription factors pivitol in regulatory functions in development and metabolism impacting organogenesis, cell proliferation, cell differentiation, inflammation and metabolism of lipids.

It has been reported that beta oxidation in peroxisomes regulates the level of arachidonic acid indirectly as a precursor of eicosanoids. Aracidonic acid is a crucial precursor and a neuroprotective. Inadequate stores of AA can compromise detoxification, which we have observed to be prevalent in our database of red cell fatty acid analyses in patients with medically diagnosed heavy metal and chemical toxicity.

Although an optimum balance of HUFAs or eicosanoids has not yet been elucidated in the literature, our database that includes 15,000 red cell fatty acid analyses, in combination with our extensive clinical data that includes detailed history of patients' oral intake and subsequent testing after aggressive supplementation of EFAs, has led us to the creation of the inventive targeted EFA balanced approach per individual red cell fatty acid analysis. A disturbed balance of HUFAs and eicosanoids appears to be unique among families complicated by various environmental exposures, digestive difficulties, especially those involving the gall bladder, and most importantly, oral access to HUFAs. Liberal access to dietary and balanced oral supplementation of HUFAs must be supplied as meat fat, evening primrose oil, cream, butter, egg yolk, and fish such as wild salmon and sardines. PUFAs, however, should also be utilized as cold pressed sunflower oil and flax oil as in the SR-3 LA to ALA ratio.

1.2.1 Omega-3 fatty acids

Alpha Linolenic Acid (ALA) is the principal Omega-3 fatty acid, which a healthy human will convert into eicosapentaenoic acid (EPA), and later into docosahexaenoic acid (DHA). Omega-3s are used in the formation of cell walls, making them supple and flexible, and improving circulation and oxygen uptake with proper red blood cell flexibility and function.

Omega-3 deficiencies are linked to decreased memory and mental abilities, tingling sensation of the nerves, poor vision, increased tendency to form blood clots, diminished immune function, increased triglycerides and “bad” cholesterol (LDL) levels, impaired membrane function, hypertension, irregular heart beat, learning disorders, menopausal discomfort, and growth retardation in infants, children, and pregnant women.

Food containing alpha linolenic acid includes flaxseed oil, flaxseed, flaxseed meal, hempseed oil, hempseed, walnuts, pumpkin seeds, Brazilian nuts, sesame seeds, avocados, some dark leafy green vegetables (e.g., kale, spinach, mustard greens, collards, etc.), canola oil (cold-pressed and unrefined), soybean oil, and others. Higher order omega 3 fatty acids (HUFA) include wild salmon, mackerel, sardines, anchovies, albacore tuna, cod liver oil, fish oil, and other cold water fish. Foods rich in higher order—HUFA omega-3 fatty acids—as wild salmon and sardines are suggested to the subjects as part of their diet.

In one embodiment, one part of alpha linolenic acid as cold pressed, organic flaxseed oil is utilized with four parts of linoleic acid omega-6 oil as cold pressed, organic sunflower oil as a 4:1 omega 6 to omega 3 ratio balanced oil.

1.2.2. Omega-6 (Linoleic Acid)

Linoleic Acid is the primary Omega-6 fatty acid. A healthy human with good nutrition will convert linoleic acid into gamma linolenic acid (GLA), which will later synthesized with EPA from the Omega-3 group into eicosanoids. Eicosanoids are hormone-like compounds, which aid in many bodily functions including vital organ function and intracellular activity.

Some Omega-6s improve diabetic neuropathy, rheumatoid arthritis, PMS, skin disorders (e.g. psoriasis and eczema), inflammation, allergies, autoimmune conditions and aid in cancer treatment. Food containing linoleic acid includes safflower oil, sunflower seed, sunflower oil, hempseed oil, hempseed, pumpkin seeds, borage oil, evening primrose oil, black currant seed oil, among many others.

In one embodiment of the invention, evening primrose oil is utilized daily as part of the therapy for autism as about 910 mg to about 2600 mg of gamma linolenic acid is contained in this oil. In another embodiment of the invention, four parts of linoleic acid omega-6 oil as cold pressed, organic sunflower oil is utilized along with 1 part of alpha linolenic acid as cold pressed, organic flaxseed oil as a 4:1 omega 6 to omega 3 ratio balanced oil.

1.3. Methylating Agents

Methylating agents donate methyl groups to molecules to enhance or reduce their expression. One important function of Methylating agents is in cellular regeneration and repair per stimulation of DNA expression. Another important function of methylating agents is to selectively “rescue” normal cells from the adverse effects of methotrexate or other poisonous substances. Other functions of methylating agents involve impeding the ability of cancer cells to divide.

Encompassed within the scope of the claimed invention are several types and classes of methylating agents. In a preferred embodiment of the invention, the methylating agent is in a natural form or derived from a natural source. Such natural methylating agents include, by way of example and not limitation, agents within the family of vitamin B group of vitamins including Methylcobalamin, Leucovorin/Folinic Acid, tetrahydrobiopterin, or a combination thereof.

Disturbances in methylation pathways may occur after exposure to heavy metals, thimerosal (preservative in vaccinations), large quantities of alcohol, or chemicals or medication (terbutaline). See, for example, in MOLECULAR ORIGINS OF HUMAN ATTENTION—THE DOPAMINE—FOLATE CONNECTION by Richard C. Deth (Kluwer Academic Publishers: Norwell, Mass., (2003)), incorporated herein by reference in its entirety. Dr. Deth, describes damage to the enzyme methionine synthase after exposure to heavy metals and alcohol whereby the enzyme may be stimulated by the use of the methylated B vitamins methylcobalamin and tetrahydrofolate or folinic acid. A direct connection between polymorphism resulted from toxic exposures to the enzyme methylene tetrahydrofolate reductase (MTHFR) has also been widely documented in the literature. If methylation pathways are not supported with methylated forms of the B vitamins folinic acid and methylcoblamin, the ability to detoxify, balance hormones, stabilize cell membrane functions, rejuvenate DNA expression, and to lock neurotransmitters such as dopamine and serotonin to their receptors is grossly impaired.

1.3.1. Methylcobalamin

Methylcobalamin is a type of Vitamin B12. Vitamin B12 has several different formulations including hydroxy, cyano, and adenosyl, but only the methyl form is used in the central nervous system. Deficiency states are fairly common and vitamin B12 deficiency mimics many other disease states of a neurological or psychological kind, and it causes anemia. B12 is converted by the liver into methylcobalamin but not in therapeutically significant amounts. Vitamin B12 deficiency is caused by a wide range of factors including low gastric acidity (common in older people), use of acid blockers such as Prilosec™ or excessive laxative use, lack of intrinsic factor, poor absorption from the intestines, lack of Calcium, heavy metal toxicity, excessive Vitamin B12 degradation, internal bleeding, excessive menstrual flow, exposure to high amounts of alcohol, or damage to methylation pathways/enzymes such as methylene tetrahydrofolate reductase (MTHFR) due to toxicity exposure, among others.

Methylcobalamin donates methyl groups to the myelin sheath that insulates nerve fibers and regenerates damaged neurons. In a B12 deficiency, toxic fatty acids destroy the myelin sheath but high enough doses of B12 can repair it. Methylcobalamin is better absorbed and retained than other forms of B12 (such as cyanocobalamin). Methylcobalamin protects nerve tissue and brain cells and promotes healthy sleep and is a cofactor of methionine synthase, which reduces toxic homocysteine to the essential amino acid methionine. Methylcobalamin also protects eye function against toxicity caused by excess glutamate.

The accumulation of VLCFAs and the resulting formation of ceramides in the brain/CNS may reflect impaired detoxification in methylation. To date every child with ASD and PDD tested for MTHFR (methylene tetrahydrofolate reductase) mutation has had a positive result for C677T, A1298C or both. The phenomenon of disturbed peroxisomal function is not limited to autism and PDD, but has been observed in our patients with ALS, MS, Parkinson's Disease, Post Stroke, AIDS, Alzheimer's, seizure disorders and toxicity states after exposure to neurotoxic environmental mold, heavy metals, methylmercury in fish, pesticides, chemicals and microbial infections.

There are striking relationships of toxic exposure (chemicals, heavy metals) and autism to disruption in methylation pathways. Impaired methylation capacity in children with autism implicates metabolic imbalance. Disturbances in methylation can result in impaired detoxification, altered genetic expression, suppressed growth and repair, poor binding of dopamine and serotonin to their receptors, which require a methyl group in their headgroup of their phospholipid for a stable connection to the cell membrane.

1.3.2. Leucovorin, Tetrahydrobiopterin, Folinic Acid

Leucovorin is the active form of the B complex vitamin, Folinic acid. Leucovorin is used as an antidote to drugs that decrease levels of Folinic Acid. Folinic Acid assists the formation of red and white blood cell and the synthesis of hemoglobin. Some treatments require what is called leucovorin rescue, because the drug used to treat the cancer or other infection has had an adverse effect on Folinic Acid levels. Leucovorin is used to reduce anemia in people taking dapsone. Leucovorin is also taken to decrease the bone marrow toxicity of sulfa drugs, and in combination with pyrimethamine to decrease the toxicity of toxoplasmosis treatment. Leucovorin is also used in combination with trimetrexate to prevent bone marrow toxicity and in combination with chemotherapeutic agents such as methotrexate. Other substituents for Leucovorin include Citrovorum, Wellcovorin, and/or folinic acid, among others.

Leucovorin calcium (Folinic acid) is a reduced form of folic acid. It is usually used 24 hours after methotrexate to selectively “rescue” normal cells from the adverse effects of methotrexate caused by inhibition of production of reduced folates. It is not used simultaneously with methotrexate, as it might then nullify the therapeutic effect of the methotrexate. More recently, leucovorin has also been used to enhance the activity of fluorouracil by stabilizing the bond of the active metabolite (5-FdUMP) to the enzyme thymidylate synthetase. Commercially available Leucovorin is the racemic mixture of D and L isomers. It is now recognized that the activity of Leucovorin is due to the L form.

In one embodiment, the treatment method of the invention comprises administration of oral folinic acid (e.g., about 1600 mcg.) and methylcobalamin (e.g. about 2 to 5 mg.) in patients with autistic spectrum disorder. Increased dosage resulted in more positive outcomes, especially along with methylcoblamin intramuscularly, Leucovorin (folinic acid), or a combination thereof. In a preferred embodiment, methylcoblamin is administered by IV infusion and Leucovorin is administered intramuscularly. By supporting methylation via methylcobalmin and folinic acid, the treatment methods of the invention amplify detoxification as well as stabilizing membrane function.

1.3.3. Synthetic Methylating Agents

Synthetic methylating agents, which impair the ability of malignant cells to divide, include dacarbazine (DTIC), temozolomide (TMZ), procarbazine, Methylnitrosourea, N-methyl-N-nitrosourea (MNU), methyl methanesulfonate (MMS) and methyl iodide, among others.

1.4 Glutathione

Reduced Glutathione (rGlutathione) is known chemically as N—(N-L-gamma-glutamyl-L-cysteinyl) glycine and is abbreviated as GSH. Its molecular formula is C10H17N3O6S and its molecular weight is 307.33 Daltons. Glutathione disulfide is also known as L-gamma-glutamyl-L-cysteinyl-glycine disulfide and is abbreviated as GSSG. Its molecular formula is C20H32N6O12S2. The term glutathione is typically used as a collective term to refer to the tripeptide L-gamma-glutamyl-L-cysteinylglycine in both its reduced and dimeric forms. Monomeric glutathione is also known as reduced glutathione and its dimer is also known as oxidized glutathione, glutathione disulfide and diglutathione. Reduced glutathione is also called glutathione and the glutathione dimer is referred to as glutathione disulfide.

Glutathione is widely found in all forms of life and plays an essential role in the health of organisms, particularly aerobic organisms. In animals, including humans, and in plants, glutathione is the predominant non-protein thiol and functions as a redox buffer, keeping with its own SH groups proteins in a reduced condition, among other antioxidant activities.

Glutathione plays roles in catalysis, metabolism, signal transduction, gene expression and apoptosis. It is a cofactor for glutathione S-transferases, enzymes which are involved in the detoxification of xenobiotics, including carcinogenic genotoxicants, and for the glutathione peroxidases, crucial selenium-containing antioxidant enzymes. It is also involved in the regeneration of ascorbate from its oxidized form, dehydroascorbate.

Glutathione functions as an antitoxin as well as antioxidant and is extremely important for the protection of major organs, the function of the immune system, and the fight against aging. It minimizes the damage caused by free radicals that is important for the health of cells. Recent, extensive research has shown the direct relationship between decreased glutathione levels and the progression of many chronic diseases. It is reported that decreased Glutathione may be a result of various types of prolonged stress and hyperactivity of the immune system, which in turn compromises the health of the body's cells. Unfortunately, taking Glutathione (L-Glutathione capsules) orally is not a suitable method for replacement of losses since the glutathione molecule is very unstable and is destroyed by the stomach acid before it can be absorbed.

Gluthathione's major effect is intracellular, and intra-organelle. Within the mitochondria Glutathione is present in tissues in concentrations as high as one millimolar. There are undoubtedly roles of glutathione that are still to be discovered.

1.5 Sodium Phenylbutyrate (PBA)

Butyrate is an important short chain fatty acid that provides fuel for colon cells and may help protect against colon cancer. The most potent dietary source of butyrate is reported to be butter (3%). Butyrate is made in the colon by bacteria. Antibiotics kill the bacteria that produce butyrate. Butyrate has a particularly important role in the colon, where it is the preferred substrate for energy generation by colonic cells.

Butyrate has been shown to significantly inhibit the growth of cancerous colon cells. Scientists have found a human gene that stops the growth of cancer cells when activated by fiber processing in the colon. Whether by supplement or by enema, a few pilot studies suggest that the presence of butyrate in colon is useful in reducing symptoms and restoring indicators of colon health in ulcerative colitis, but one study showed no benefit over placebo. Several doctors claim that many people are helped with butyrate enemas. Butyrate levels are commonly measured in comprehensive stool analyses and act as a marker for levels of beneficial bacteria.

One possible mechanism of action of butyrate is through breaking up ceramides which accumulate in the membrane as clusters called “lipid rafts”. Rafts are composed of ceramides, cholesterol and sphingomyelin (SM) all of low energy with either very long chains or rigid chains (e.g. cholesterol.) Ceramides are generally structured with lipid tails as very long chain fatty acids (VLCFAs) and combine with PC to form SM (reversible back into ceramide and phosphatidylcholine). SM maintains the VLCFAs from the ceramide as opposed to holding on to the former high active lipids formerly associated with PC. Most diseases and aging tends towards a higher concentration of raft formation. This is complicated with signaling emanating from rafts that encourages apoptosis, which is both destructive and constructive.

The low activity level of the three lipids encourages the agglomeration into rafts which ultimately degrades the fluidity of vibrant active membranes. Most diseases and aging tend towards a higher concentration of raft formation. This is complicated with signaling emanating from rafts that encourage apoptosis, which is both destructive and constructive.

Although scientists have long linked butyrate to overall reductions in the incidence of colon cancer, the molecular basis of that benefit has remained largely unknown. Butyrate affects a chemical that otherwise bind and constrict the activity of the p21 gene that is involved in the growth of cancer cells. Butyrate optimizes itself in the body. Concentrations of butyrate in the composition of the invention can range from about 1-10 grams per liter or more, depending on the specific condition at hand. Minamiyama et al. Hum. Mol. Genet. 1:13(11):1183-92 (2004), (incorporated herein by reference by its entirety) in a study using mouse model of Bulbar ALS, demonstrated oral administration of sodium butyrate (SB) successfully ameliorated neurological phenotypes as well as increased acetylation of nuclear histone in neural tissues.

When β-oxidation of Renegade fatty acids is impaired, sodium phenylbutyrate (PBA) is used that is a short chain fatty acid and has a long clinical history of treatment for hyperammonemia and urea cycle disorders (ornithine transcarbamylase deficiency) without adverse effects. The use of sodium phenylbutyrate or calcium/magnesium butyrate, a short 4-carbon chain fatty acid, is of striking benefit in breaking apart and mobilizing renegade fats, lowering glutamate and aspartate, affecting neuronal excitability, sequestering ammonia, clearing biotoxins, preventing cerebral ischemic injury, acting as a histone deacetylase inhibitor as well as having neuroprotective effects.

In ALS and ASD models PBA addresses the formation of lipid rafts, and neuroinflammation as well as having neuroprotective effects as a histone deacetylase inhibitor and prolonging survival and regulating expression of anti-apoptotic genes. PBA inhibits the induction of iNOS (inducible nitric oxide synthase) and proinflammatory cytokines such as tumor necrosis factor alpha in astrocytes, microglia and macrophages implicating a neuroprotective role. PBA has also been shown to suppress the proliferation of myelin basic protein primed T cells and may inhibit the disease process of experimental allergic encephalomyelitis.

In one embodiment of the invention, there is provided treatment methods and compositions containing PBA. The adult patients with ALS have demonstrated marked positive responses to intravenous use of sodium phenylbutyrate. The pediatric patients have used both the IV sodium phenylbutyrate and oral phenylbutyrate (e.g., 1 gram to 4 grams IV) for several years with a dosage of 1, 2, 3, 4, 5, or 6 grams daily. Prior to the introduction of phenylbutyrate, membrane lipid stabilization must be achieved with essential fatty acids and phosphatidylcholine. The aggressive use of IV sodium phenylbutyrate without essential fatty acids and PC leads to clinical instability in adult patients with ALS.

1.6 Electrolytes

Electrolyte is a “medical/scientific” term for salts, specifically ions. The term electrolyte means that ion is electrically-charged and moves to either a negative (cathode) or positive (anode) electrode. Electrolytes are vital elements of a healthy body and are needed for the proper performance of bodily organs and tissues by maintaining the voltages across the cell membranes and to carry electrical impulses (nerve impulses, muscle contractions) across these cells and to other cells. The kidneys function is to keep the electrolyte concentrations constant in the blood despite changes in the body. For example, during a heavy exercise the body loses electrolytes in the sweat, particularly sodium and potassium. These electrolytes must be replaced to keep the electrolyte concentrations of the body fluids constant. So, many sports drinks have sodium chloride or potassium chloride added therein.

The types of electrolytes used within the scope of the invention include, by way of example and not limitation, sodium (Na⁺), potassium (K⁺), chloride (Cl⁻), Calcium (Ca²), Magnesium (mg²), bicarbonate (HCO₃⁻), Phosphate (PO₄⁻²) and sulfate (SO₄⁻²), among others.

1.7 Trace Minerals

Another important constituent of the pharmaneutical composition of the invention as described herein includes trace minerals. Suitable mineral compositions include solid multi-mineral preparations, or the E-Lyte Liquid Mineral™ set #1-8 (separate solutions of biologically available potassium, zinc, magnesium, copper, chromium, manganese, molybdenum, and selenium, or a combination thereof, or #1-9 (separate solutions of biologically available potassium, zinc, magnesium, copper, chromium, manganese, molybdenum, selenium and iodine), or a combination thereof. Both E-Lyte Liquid Mineral™ set #1-8, and E-Lyte Liquid Mineral™ set #1-9 set are available from E-Lyte, Inc. (Millville, N.J., USA).

2. Pharmaneutical Compositions

The present invention provides pharmaneutical compositions comprising a therapeutically effective amount of a first composition comprising one or more phosphotidylcholine formulations and the second composition comprising one or more constituents comprising essential fatty acid supplements, trace minerals, butyrate, electrolytes, methylating agents (methylcobalamin, folinic acid/Leucovorin), glutathione, or a combination thereof, in a suitable carrier.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In general, the combinations may be administered by the transdermal, intraperitoneal, intracranial, intracerebroventricular, intracerebral, intravaginal, intrauterine, oral, rectal, ophthalmic (including intravitreal or intracameral), nasal, topical (including buccal and sublingual), parenteral (including subcutaneous, intraperitoneal, intramuscular, intravenous, intradermal, intracranial, intratracheal, and epidural) administration.

A typical regimen for preventing, suppressing, or treating a disease or disored related to an imbalance of essesntial fatty acids comprises administration of an effective amount of the composition as described above, administered as a single treatment, or repeated as enhancing or booster dosages, over a period up to and including one week to about 48 months or more.

The pharmaneutical compositions of the present invention, suitable for inoculation or for parenteral or oral administration, are in the form of sterile aqueous or non-aqueous solutions, suspensions, or emulsions, and can also contain auxiliary agents or excipients that are known in the art.

In one embodiment, the composition is formulated in accordance with routine procedures adapted for intravenous administration to human beings. Typically, compositions for intravenous administration are solutions in sterile isotonic aqueous buffer. Where necessary, the composition may also include a solubilizing agent and a local anesthetic such as procaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water (not saline). Where the composition is administered by injection, an ampoule of sterile water for injection or saline can be provided so that the ingredients may be mixed prior to administration.

In addition, the compositions of the invention may be incorporated into biodegradable polymers allowing for sustained release of the compound, the polymers being implanted in the vicinity of where the delivery is desired, so that the composition is slowly released systemically.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The pharmaneutical composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Within other embodiments, the compositions may also be placed in any location such that the compounds or constituents are continuously released into the aqueous humor. The amount of the composition of the invention which will be effective in the treatment, inhibition and prevention of Autism can be determined by standard clinical techniques. In addition, in vitro assays may optionally be employed to help identify optimal dosage ranges.

In particular, the dosage of the compositions of the present invention will depend on the disease state of Autism and other clinical factors such as weight and condition of the human or animal and the route of administration of the compounds or compositions. The precise dose to be employed in the formulation, therefore, should be decided according to the judgment of the health care practitioner and each patient's circumstances. Effective doses may be extrapolated from dose-response curves derived from in vitro or animal model test systems.

Treating humans or animals between approximately 0.5 to 500 mg/kilogram is a typical broad range for administering the pharmaneutical composition of the invention. The methods of the present invention contemplate single as well as multiple administrations, given either simultaneously or over an extended period of time.

Preferred unit dosage formulations are those containing a daily dose or unit, daily sub-dose, or an appropriate fraction thereof, of the administered compositions. It should be understood that in addition to the compositions, particularly mentioned above, the formulations of the present invention may include other agents conventional in the art having regard to the type of formulation in question.

The pharmaneutical composition of the invention comprises a dry formulation, an aqueous solution, or both. Effective amounts of a phosphatidylcholine composition, EFA composition, trace minerals, rglutathione, butyrate, electrolytes, or methylating agents (methylcobalamin, Leucovorin/folinic acid) can each be formulated into the pharmaneutical composition for treating autism or for delaying the onset of autism symptoms in a subject. As used herein, a “pharmaneutical composition” includes compositions for human and veterinary use. Pharmaneutical compositions for parenteral (e.g., intravascular) administration are characterized as being sterile and pyrogen-free. One skilled in the art can readily prepare pharmaneutical compositions of the invention for enteral or parenteral use, for example by using the principles set forth in Remington's Pharmaceutical Science, 18^{th} edit. (Alphonso Gennaro, ed.), Mack Publishing Co., Easton, Pa., 1990.

Because phosphatidylcholine, linoleic acid and alpha linolenic acid are all soluble in oils or lipids, they can be conveniently formulated into a single pharmaneutical composition. Thus, in one embodiment, the invention provides a single-dose pharmaneutical composition comprising a phosphotidylcholine composition and an EFA 4:1 composition. Those constituents that are water soluble, such as for example, the liquid trace minerals, and electrolytes are generally not formulated into a single pharmaneutical composition with the phosphatidylcholine and EFAs compositions, but are rather formulated as separate compositions. However, the water soluble constituents, the phosphatidylcholine composition, and the EFA composition can be formulated into a single pharmaceutical composition as an emulsion, for example an oil-in-water emulsion or water-in-oil emulsion.

The pharmaneutical compositions of the invention can be in a form suitable for oral use, according to any technique suitable for the manufacture of oral pharmaceutical compositions as are within the skill in the art. For example, the phosphatidylcholine composition and the EFA composition can be formulated (either separately or together) into soft capsules, oily suspensions, or emulsions, optionally in admixture with pharmaceutically acceptable excipients. Suitable excipients for a phosphatidylcholine composition or EFA composition comprise oil-based media; e.g., archis oil, liquid paraffin, or vegetable oils such as olive oil. Butyrate is administered in encapsulated form, for example, as Magnesium/Calcium Butyrate from BodyBio, Inc., (Millville, N.J., USA) or Sodium Phenylbutyrate from Triple Crown America (Perkasie, Pa., USA) or as IV Liquid Sodium PhenylButyrate from Wellness Health and Pharmaceuticals (Birmingham, Ala., USA).

The compositions of the invention are formulated into liquid or solid compositions, such as aqueous solutions, aqueous or oily suspensions, syrups or elixirs, emulsions, tablets, dispersible powders or granules, hard or soft capsules, optionally in admixture with pharmaceutically acceptable excipients.

2.1. Adjuvants, Carriers, and Diluents

As would be understood by one of ordinary skill in the art, when a composition of the present invention is provided to an individual, it can further comprise at least one of salts, buffers, adjuvants, or other substances which are desirable for improving the efficacy of the composition. Adjuvants are substances that can be used to specifically augment at least one immune response. Normally, the adjuvant and the composition are mixed prior to presentation to the immune system, or presented separately.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Adjuvants can be generally divided into several groups based upon their composition. These groups include lipid micelles, oil adjuvants, mineral salts (for example, AlK(SO₄)₂, AlNa (SO₄)₂, AlNH₄ (SO₄)), silica, kaolin, and certain natural substances, for example, wax D from Mycobacterium tuberculosis, substances found in Corynebacterium parvum, or Bordetella pertussis, Freund's adjuvant (DIFCO), alum adjuvant (Alhydrogel), MF-50 (Chiron) Novasomes™, or micelles, among others.

Suitable excipients for liquid formulation include water or saline, suspending agents such as sodium carboxymethylcellulose, methylcellulose, hydroxypropylmethyl-cellulose, sodium alginate, polyvinylpyrrolidone, gum tragacanth, and gum acacia; dispersing or wetting agents such as lecithin, condensation products of an alkylene oxide with fatty acids (e.g., polyoxethylene stearate), condensation products of ethylene oxide with long chain aliphatic alcohols (e.g., heptadecethyleneoxy-cetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyoxyethylene sorbitan monooleate).

Suitable excipients for solid formulations include calcium carbonate, sodium carbonate, lactose, calcium phosphate, or sodium phosphate; granulating and disintegrating agents such as maize starch, or alginic acid; binding agents such as starch, gelatin, or acacia; and lubricating agents such as magnesium stearate, stearic acids, or talc, and inert solid diluents such as calcium carbonate, calcium phosphate, or kaolin.

Other suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

Oral pharmaneutical compositions of the invention can contain one or more agents selected from the group consisting of sweetening agents, flavoring agents, coloring agents, and preserving agents in order to provide a pharmaneutically palatable preparation.

Liquid formulations according to the invention can contain one or more preservatives such as ethyl, n-propyl, or p-hydroxy benzoate; one or more coloring agents; one or more flavoring agents; or one or more sweetening agents such as sucrose, saccharin, or sodium or calcium cyclamate.

Liquid pharmaceutical formulations according to the invention, especially those comprising a phosphotidylcholine composition or an EFA composition can contain antioxidants such as tocopherol, sodium metabisulphite, butylated hydroxytoluene (BHT), butylated hydroxyanisole (BHA), ascorbic acid or sodium ascorbate.

The pharmaneutical compositions of the invention are in the form of sterile, pyrogen-free preparations suitable for parenteral administration, for example as a sterile injectable aqueous solution, a suspension or an emulsion. Such pharmaneutical compositions can be formulated using the excipients described above for liquid formulations. For example, a sterile injectable preparation according to the invention can comprise a sterile injectable solution, suspension or emulsion in a non-toxic, parenterally-acceptable diluent or solvent; e.g., as a solution in 1,3-butanediol, water or saline solution. Formulations of sterile, pyrogen-free pharmaneutical compositions suitable for parenteral administration are within the skill in the art.

3. Methods of Treating Autism

A subject presenting with symptoms indicative of autism can be treated by the methods and compositions of the invention to prevent, delay, ameliorate or treat one or more symptoms of autism symptoms. The “treatment” provided need not be absolute, i.e., the autism need not be totally prevented or treated, provided that there is a statistically significant improvement relative to a control population. Treatment can be limited to mitigating the severity or rapidity of onset of symptoms of the disease.

A typical regimen for preventing, suppressing, or treating a disease or condition related to autism comprises administration of an effective amount of the composition as described above, administered as a single treatment, or repeated as enhancing or booster dosages, over a period up to and including one week to about 48 months or more, or permanently if it need be.

The compositions of the invention can be administered to the subject by any parenteral or enteral technique suitable for introducing the composition into the blood stream or gastrointestinal tract, including intravascular (e.g., intravenous and intraarterial) injection and oral administration. In a preferred embodiment, one or more compositions are administered to the subject both by mouth, intravascularly, or both.

An “effective amount” of the compositions of the invention is any amount sufficient to therapeutically inhibit the progression of autism, or to prophylactically delay the onset of autism symptoms. For example, the concentration of phosphatidylcholine in a composition can range from about 500 mg to about 10,000 mg or more, about 6000 mg to about 7500 mg, from about 2000 to about 5000 mg, and from about 3000 mg to about 4000 mg phosphatidylcholine. It is intended herein that by recitation of such specified ranges, the ranges recited also include all those specific integer amounts between the recited ranges. For example, in the range of about 3000 mg to 4000 mg, it is intended to also encompass 3200 mg to 43000 mg, 3300 mg to 3800 mg, etc, without actually reciting each specific range therewith. Phosphatidylcholine compositions can be administered intravenously, orally, or both.

One of ordinary skill in the art can readily determine an appropriate temporal and interval regimen for administering the compositions of the invention. For example, the compositions of the invention can be administered once, twice or more daily, for one, two, three, four, five, six or seven days in a given a week, for one or several weeks or months. The length of time that the subject receives the composition can be determined by the subject's physician or other health care providers and caretakers, according to need. Due to the chronic and progressive nature of autism, it is expected that subjects will receive one or more compositions according to the present methods for an indefinite period of time, likely for the rest of their lives.

In one embodiment of the invention, a phosphatidylcholine composition containing about 500 mg to 1000 mg phosphatidylcholine is administered to a subject intravenously, for example two to three times daily, for consecutive or non-consecutive days in a given week. Another phosphatidylcholine composition which contains about 3600 mg to about 18,000 mg phosphatidylcholine is administered, for example once or twice, to the same subject daily by mouth.

In another embodiment, one or more compositions comprising linoleic acid and alpha linolenic acid in an approximately 4:1 (v/v) ratio are administered to a subject who has been diagnosed with, or has demonstrated one or more symptoms of autism. Linoleic acid, and alpha linolenic acid, can be administered separately to a subject, as long as the ratio (v/v) of linoleic acid to alpha linolenic acid administered within a given time frame (e.g., 24 hours or less, 12 hours or less, 6 hours or less, or 4 hours or less) is approximately 4:1. The term “EFA 4:1 composition” therefore refers to one or more compositions comprising linoleic acid and one or more compositions comprising alpha linolenic acid, which are administered separately or together to a subject at about 4:1 (v/v) ratio of linoleic acid to alpha linoleic acid.

Any commercially available preparation comprising linoleic acid and alpha linolenic acid, or mixtures of the two in an approximately 4:1 (v/v) ratio, can be used as the EFA 4:1 composition in the present methods. Suitable EFA 4:1 compositions include the BodyBio Balance 4:1™ EFA oil available from BodyBio Inc. (Millville, N.J. USA), or any mixtures containing the essential fatty acids, such as for example, a mixture of cold pressed organic safflower or sunflower oil and flaxseed oil to yield a 4:1 ratio of linoleic acid to linolenic acid (4 parts Omega 6: to 1 part Omega 3).

The EFA compositions can be administered to a subject by any parenteral or enteral technique suitable for introducing the EFA composition into blood stream or the gastrointestinal tract. In a preferred embodiment, the EFA 4:1 compositions are administered to the subject by mouth.

An “effective amount” of EFA 4:1 compositions is any amount sufficient to inhibit the progression of autism, or to delay the onset of autism symptoms, when administered in conjunction with the phosphatidylcholine and one or more compositions containing trace minerals, rglutathione, butyrate, electrolytes, methylating agents (folinic acid, methylcobalamin), or a combination thereof. For example, an effective amount of the EFA 4:1 composition can be from about 10 mls (about 2 teaspoons) to about 100 mls (about 7 tablespoons), about 15 mls (about 1 tablespoon) to about 80 mls (about 5 tablespoons), or about 30 mls (about 2 tablespoons) to about 60 mls (about 4 tablespoons).

One skilled in the art can readily determine an appropriate dosage regimen for administering the EFA compositions. For example, the EFA compositions can be administered once, twice or more daily, for one, two, three, four, five, six or seven days in a given week. The length of time that the subject receives EFA compositions can be determined by the subject's physician according to need. According to the severity of the symptoms of autism and its chronic or progressive nature, subjects may be expected to receive EFA compositions according to the present methods for an indefinite period of time, likely for the rest of their lives.

In one embodiment, about 30 mls to about 60 mls (about 2 to about 4 tablespoons) of the EFA 4:1 composition is administered to a subject by mouth, once to twice daily.

In another embodiment, gamma linolenic acid is administered by mouth as evening primrose oil from about 910 mg to about 2600 mg.

In the practice of the present methods, an effective amount of compositions comprising trace minerals are administered to subject who has been diagnosed with, or who is at risk for developing autism. The trace minerals in one or more same or different compositions are administered to the subject, or two or more mineral compositions can be administered separately. It is understood that mineral compositions can be administered separately to a subject, as long as the compositions are administered within a given time frame (e.g., 24 hours or less, preferably 12 hours or less, more preferably 6 hours or less, particularly preferably 4 hours or less). Preferably, mineral compositions for use in the present methods comprise biologically available forms of potassium, magnesium, zinc, copper, chromium, manganese, molybdenum, selenium, iodine, or any combination thereof, although the mineral compositions can comprise other minerals in biologically available form.

The compositions comprising trace minerals can be administered to a subject by any parenteral or enteral technique suitable for introducing the compositions into the blood stream or gastrointestinal tract. In one embodiment, the compositions comprising trace minerals are administered to the subject by mouth.

Also encompassed within the scope of the invention is the use of the electrolytes. In one embodiment, a balanced electrolyte concentrate is administered orally with one to fifteen tablespoons diluted in fluid. E-Lyte Balanced Electrolyte is a concentrated high K:Na ratio solution that is usually diluted with H₂O at 16:1. In another embodiment the subject is instructed to take the electrolyte in its concentrated form, one to three tablespoons at a time followed by 1 or 2 ounces of H₂O, throughout the day.

Any commercially available composition or compositions comprising one or more biologically available minerals can be used as trace mineral composition of the present invention. Suitable mineral compositions include solid multi-mineral preparations, or the E-Lyte Liquid Mineral™ set #1-8 (separate solutions of biologically available potassium, zinc, magnesium, copper, chromium, manganese, molybdenum, and selenium) or #1-9 (separate solutions of biologically available potassium, zinc, magnesium, copper, chromium, manganese, molybdenum, selenium and iodine), both available from E-Lyte, Inc. (Millville, N.J. USA).

The effective amount of the trace minerals is determined for each subject according to that subject's needs and nutritional status, based on a nutritional evaluation of the subject. Suitable techniques for performing a nutritional evaluation of a subject include standard blood tests to determine serum mineral and electrolyte levels, and subjective evaluations such as the E-Lyte, Inc. “taste test” for determining mineral deficiencies. The E-Lyte, Inc. “taste test” for determining mineral deficiencies is described below in the Examples.

After determining the effective amount of the one or more mineral compositions for administration to the subject, one skilled in the art can readily determine the dosage regimen for administering mineral compositions. For example, the trace minerals can be administered once, twice or more daily, for one, two, three, four, five, six or seven days in a given week. Preferably, the one or more mineral compositions are administered to the subject twice a day, for seven days in a given week. The length of time that the subject receives the mineral compositions can be determined by the subject's physician or primary caretaker, according to need. Due to the chronic and progressive nature of Autism, it is expected that subjects will receive the one or more mineral compositions according to the present methods for an indefinite period of time, likely for the rest of their lives.

In another embodiment, a subject being treated according to the present methods receives intravascular (e.g., intravenous) reduced Glutathione. For example, a subject can receive from about 1000 mg to about 3000 mg of rglutathione, about 1500 mg to about 2800 mg rGlutathione, about 1800 mg to about 2400 mg rGlutathione, once, twice or more daily, for one, two, three, four, five, six or seven days a week. In one embodiment, the subject receives about 1800 mg to about 2400 mg intravenous rGlutathione twice daily, for three consecutive or non-consecutive days in a given week. In another embodiment, the rglutathione is administered in reduced form as an intravenous “fast push” over three to five minutes.

Any commercially available composition comprising rglutathione can be used in the present methods. Suitable compositions comprising rglutathione include the rGlutathione preparations from Wellness Health and Pharmaceuticals (Birmingham, Ala., USA) or Medaus Pharmacy (Birmingham, Ala., USA).

It is also preferable to maintain a subject being treated by the present methods on a low carbohydrate, high protein, high green vegetable, high legume as butter beans/mucuna, high fat diet termed the Detoxx Diet, e.g., a diet excluding all grains, sugars, fruit, fruit juices, all “below ground” root vegetables and processed foods. Suitable low carbohydrate, high protein, high fat diets include such well-known diets as Atkins® or the South Beach Diet™ (see, e.g., Atkins RC, Atkins for Life, St. Martins Press, NY, 2003 and Agatston A, THE SOUTH BEACH DIET: THE DELICIOUS, DOCTOR-DESIGNED, FOOLPROOF PLAN FOR FAST AND HEALTHY WEIGHT LOSS, Random House, N.Y., 2003, the entire disclosures of which are herein incorporated by reference). A diet lower in carbohydrate suppresses phospholipase A2 (PLA2), an enzyme that stimulates the catalyzing or breaking apart of the essential fatty acids from the phospholipids in the cell membrane, thereby de-stabilizing the membrane and control of cellular function.

Oral support with neurotransmitter precursors is helpful with the amino acids tryptophan, theonine, mucuna beans, butter beans, tyrosine, and phenylalanine as indicated by testing of urinary neurotransmitters.

In one embodiment, the subject being treated for autism receives rGlutathione as well as phosphatidylcholine and Leucovorin, which are administered intravenously and methylcobalamin is administered by injection. This treatment regimen is termed the PK Protocol.

In another embodiment, the present methods comprise treating a subject who has been diagnosed with autism, or who is at risk for developing one or more symptoms of autism, for an indefinite period of time (e.g., five weeks or more) by:

1) intravenous administration by lipid exchange of a phosphatidylcholine (PC) composition comprising about 250 mg to about 500 mg phosphatidylcholine (e.g., bolus PC of 2 to 5 grams), followed by intravenous administration of Leucovorin, folinic acid at about 5 mg to 10 mg, and as the third part of the infusion about 1800 mg to about 2400 mg of rglutathione, twice to three times daily for a minimum 3 to 5 days in a seven-day period;

2) once or twice daily oral administration of a PC composition comprising about 3600 to about 7200 mg of phosphatidylcholine, twice daily oral administration of butyrate as 5 capsules twice daily of Magnesium/Calcium Butyrate in capsule form or 3 Tablespoons or about 45 mls of liquid phenylbutyrate twice daily and/or IV administration of sodium phenylbutyrate as 5 to 10 grams;

3) once daily oral administration of an effective amount of one or more mineral compositions, (the effective amount of the one or more mineral compositions can be doubled or tripled); and

4) once daily oral administration of about 30 mls to about 60 mls (about 2 to about 4 tablespoons) of an EFA 4:1 composition. (The 4:1 oil can be administered as above 2 to 4 times daily as determined by the subject's physician or primary caretaker).

Also encompassed within the scope of the invention is the use of the methods and compositions of the invention in combination with other commonly used treatments, and/or medications for treating ASD, so long as such combination therapies do not impair the empirical healthy nutrient balance of the individual, which balance has been restored and maintained by the pharmaneutical compositions of the invention.

5. Test Kits

The invention also provides a pharmaneutical pack or kit comprising one or more containers filled with one or more compositions or the ingredients of the pharmaneutical compositions of the invention. The kits are provided for the treatment of the symptoms of disease and disorders related to an imbalance of essential fatty acids and cell membrane dysfunction. The kit comprises instructions for treating the disease or disorder in a subject and one or more of the following components: 1) a phosphatidylcholine composition; 2) an EFA 4:1 composition; 3) mineral compositions, 4) electrolyte compositions; 5) methylating agents, methylcobalamin and folinic acid/Leucovorin; 6) rglutathione; 7) butyrate or phenylbutyrate, or a combination thereof.

If a particular component is not included in the kit, the kit can optionally comprise information on where to obtain the missing component, for example an order form or uniform resource locator for the internet specifying a website where the component can be obtained.

The instructions provided with the kit describe the practice of the methods of the invention as described above, and the route of administration and effective concentration and the dosing regimen for each of the compositions provided therein.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. The contents of all references, patents and published patent applications cited throughout this application are expressly incorporated herein by reference.

### EXAMPLES

### Example 1

### Treatment of Autistic Spectrum Disorders with Oral and IV Lipid Therapy

The case studies represented below present the result of the treatment regimen on 300 subjects studied. Phospholipid re-modeling of these subjects were stimulated by supplying oral or IV phospholipids, principally PC, as well as balanced essential fatty acids and catalysts via nutritional and phamaneutical interventions.

### Case Study One

Five year old female presented with global dyspraxia involving both gross and fine motor difficulties, underweight, small for chronological age, visual dysfunction, hypotonia, microcephaly, frozen facies, hyperteleorism, expressive language deficit, poor social interaction, disturbed balance, abnormal gait, severe irritability, learning problems, IQ of 74, anxiety, poor concentration, delay in pragmatic speech, slow progress of myelination per MRI. Patient had methylmercury exposure during fetal development as mother consumed one to two cans of white albacore tuna daily throughout the pregnancy.

Development up to six months of age within normal limits, with delay in meeting developmental milestones thereafter. Patient had abnormal subetlomere FISH (fluorescent in situ hybridization) results with a deletion of the terminal long arm region of one chromosome 3. The 3q subtelomere probe was not observed on any other chromosome, and no subtelomere probe from another chromosome was observed on the abnormal chromosome 3 thus the abnormality was read as a deletion and not a derivative chromosome. Both parents were tested and did not have the deletion or any other chromosomal abnormality. Laboratory workup revealed a buildup of renegade fatty acids with moderate suppression of omega 6 and omega 3 fatty acids in the red cell analysis, acidosis, electrolyte disturbance, gross elevation of RDW, hyperammonemia.

After six months of IV and oral therapy, with phosphatidylcholine, EFAs, folinic acid, methylcobalamin, mitochondrial/peroxisomal cocktails (thiamin, riboflavin, pyridoxine, biotin, pantothenic acid, NADH, carnitine, CoQ10), trace minerals, electrolytes, sodium phenylbutyrate and a nutrient dense PLA2 suppressive diet, patient made significant strides in learning, coordination, language, concentration, affection towards her parents and social play with peers. After 14 months of oral and IV lipid therapy patient entered a normal first grade. IQ is now measuring within the normal range and patient has excelled in her academic and social performance. Physical movements are more organized as gross and fine motor skills have developed, there has been increased growth, speech is much improved and mood is stable.

### Case Study Two

Seven year old male diagnosed with ASD at age 4 with moderate progression of autistic features. Patient presented with hyperactivity, poor motor skills, social deficits, speech delay, poor attention, hypersensitive hearing, hypotonia, poor memory, mood swings, apathy, brain fog, impulsiveness, rage behavior, unable to accomplish math skills, pica, sleep disturbance, decreased eye contact, constipation, dry skin, recurrent sinus infections, dry skin, low weight and growth over the past 3 years. Laboratory workup revealed a buildup of renegade fatty acids with decreased myelination biomarkers, suppression of omega 6 fatty acids and low total lipid content in the red cell analysis, acidosis, immune suppression with low WBC (white blood cells) and globulin, hypoglycemia, electrolyte disturbance, and hyperammonemia.

Patient had previously been given adult doses of Paxil and Effexor for three years per a physician specializing in autism. Patient was detoxified of the medication by the use of inventive intravenous phosphatidylcholine by lipid exchange followed by glutathione fast push twice weekly in our clinic. With a step down procedure the medications were completely removed over 8 weeks and the patient's mood and behavior stabilized. As infusions and oral nutrient therapy were continued over the next six months patient gained 12 pounds and grew 2 inches. His motor skills improved as did his eye contact, reading, math, sleep and behavior. Patient began to smile and laugh for the first time, able to cry producing tears, developed independence (‘I want to do it myself’), began to interact with friends and family. After one year patient continues to respond to lipid infusions and high dose oral essential fatty acid therapy.

### Case Studies Three and Four

Three year old fraternal twins with ASD and PDD presented as Twin A with severe speech delay, hypotonia, poor eye contact, not toilet trained, diarrhea, short attention span, no pointing, refusal to eat, underweight, small for chronological age. Twin B presented with hyperactivity, poor eye contact, echolalia, garbled speech, constipation, restricted food intake, underweight, small for chronological age. Twins are the product of an uncomplicated pregnancy, full term delivery with 7 pound birth weights. In the neighborhood where the twins live there is a high incidence of ASD/PDD and their home is built over what has been identified to be a site of potentially toxic material as reported by their father, a surgeon.

Twin A was found to have compound heterozygous MTHFR (methylene tetrahydrofolate reductase) mutations for C677T and A1298C while Twin B was positive for one copy of the A1298C mutation. Laboratory workup on Twin A revealed a marked decrease in myelination markers (DMAs or dimethylacetals), a buildup of renegade fatty acids with suppression of ω3 fatty acids in the red cell analysis, acidosis, increased liver enzymes, electrolyte disturbance, and hyperammonemia. Laboratory workup on Twin B also revealed a marked decrease in myelination markers (DMAs or dimethylacetals), a gross buildup of renegade fatty acids with suppression of ω6 and ω3 and low total lipid content in the red cell analysis, acidosis, increased liver enzymes, electrolyte disturbance, dehydration and low globulin. Oral supplementation was started slowly due difficulties with poor dietary intake. Food intake was improved by adding egg protein and essential fatty acids to foods the twins enjoyed. Infusions were given weekly with lipid exchange of phosphatidylcholine, leucovorin and rGSH. After the fourth infusion Twin A began speaking in full sentences, playing hide and seek, giving excellent eye contact and told his father, ‘I want you play with me!’

Twin B also had strong gains in communication and social interaction but did not receive as many oral supplements as Twin A. Both twins had marked improvement in their presentation entering a normal pre-school three months (Twin B) and six months (Twin A) after initiation of IV nutrient therapy. The twins were re-examined eight months after starting oral/IV nutrient therapy and both demonstrated striking improvement in their evaluations. Twin B has complete resolution of ASD/PDD symptoms while Twin A has some mild residual symptoms remaining. More complex mutation of MTHFR in Twin A was noted.

### Case Study Five

Nine year old female with mild ASD diagnosed at age 4 who presented with poor attention, mood swings, rage, oppositional behavior, brain fog, tan stool, blurred vision, insomnia, alternating diarrhea and constipation, learning problems, poor memory, impaired reciprocal conversation skills, delayed response to questions, poor socialization skills, difficulty interpreting social cues, underweight, small for chronological age. Laboratory workup revealed a buildup of renegade fatty acids with deep suppression of omega 6 fatty acids in the red cell analysis, acidosis, increased liver enzymes, electrolyte disturbance, hyperammonemia, low normal IGF-I (insulin growth factor reflective of methionine synthase function), positive for one copy of the MTHFR A1298C mutation and positive for toxic mold antibodies stachybotrys, herbarium and fumigatus. Patient had exposure to neurotoxic mold in the basement of her home at 3.5 years prior to appearance of symptoms of autism.

After first lipid exchange and glutathione infusion patient experienced a dramatic change with increased attention, alertness, and more stable mood. Oral therapy included high dose phosphatidylcholine, EFAs, folinic acid, methylcobalamin, riboflavin and a nutrient dense PLA2 (low refined carbohydrate, high fat and protein) suppressive diet.

After the seventh infusion parents reported that the patient was no longer angry or irritable, memory had improved, there was increased alertness, better compliance, faster verbal response to questions asked, began asking ‘why?’ and ‘can I?’ as pragmatic communication has developed, more normal social interaction with peers, schoolwork improved, less GI problems, better sleep and happier mood overall. Patient then received two doses of bolus phospholipids as 2 grams, then 3 grams on consecutive weeks dripped over 3 hours resulting in increased awareness/communication. A drip of IV Phenylbutyrate of 1.5 grams was then given the next week over 3 hours along with lipid exchange, Leucovorin, GSH before and after the drip resulting in reduced anxiety, a marked increase in verbal expression of thoughts and feelings and improved social interaction with peers.

### Case Study Six

Seven year old male with mild PDD diagnosed at age 4 who presented with severe fatigue, loss of abstract thinking, anxiety, daily headaches, apathy, depression, excessive sleepiness, irritability, impulsiveness, poor attention, mood swings, screaming/crying episodes followed by vomiting, oppositional behavior, brain fog, excessive thirst, tan stool, learning problems, poor memory, nightmares, dry skin, pale, alopecia, muscle pain, shortness of breath upon exertion, orange palms of hands/soles of feet, poor eye contact, bleeding gums, bruising easily, head banging. Patient has a normal twin, both prematuraley being born by 4 weeks. Patient had 30 ear infections starting at one month of age accompanied with liberal use of antibiotics and acetaminophen. WBC was so suppressed by the age of two that a bone marrow transplant was considered. Patient had Mono at 3.5 years and large daily doses of acetaminophen were used for one month at that time. Patient did develop fairly normally but had frequent recurrent illness and mild PDD. Patient was given 500 mg of N-acetyl cysteine (NAC) intravenously from October 2004 through March 2005 for 20 treatments which resulted in the appearance of autistic symptoms.

Patient was no longer able to perform academically as he had prior to the NAC infusions. Our laboratory workup revealed a buildup of renegade fatty acids with suppression of ω6 (DGLA) and ω3 (DHA) fatty acids along with suppression of nervonic acid (myelin precursor) in the red cell fatty acid analysis, severe hyperammonemia, acidosis, increased liver enzymes (LDH, SGOT), decreased WBC, electrolyte disturbance, low normal IGF-I (insulin growth factor reflective of methionine synthase function), positive for one copy of the MTHFR A1298C mutation, positive for previous exposure as IgG to HHV6, EBV and Strep with IgM+ to Babesia, elevation of Retinol after 6 month overdose of oral Vitamin A, elevated creatine kinase 134, and positive for toxic mold antibodies stachybotrys, tenuis, herbarium and fumigatus.

Patient was responsive to IV lipid exchange with 250 mg phosphatidylcholine which was initially given once weekly along with targeted supplementation and nutrient dense diet. IV phosphatidylcholine dose was increased to 500 mg twice weekly and after 6 weeks there was improvement in fatigue, stable mood, return of focus, improvement in memory, clearance of orange color on palms/soles, less headaches, improved learning. Glutathione was added to the IV regime after 6 weeks. Patient was given a bolus dose of 1.5 grams of phosphatidylcholine diluted in D5W dripped over 2 hours followed by glutathione which resulted in improved cognition, increased circulation, improved eye contact and more demonstrative, loving behavior. A bolus dose of 3 grams of phosphatidylcholine dripped over 4 hours was also well tolerated and patient had similar positive responses as he had with the first bolus. Patient is now attending public school in a normal classroom with his twin and is doing exceptionally well academically and socially.

### Case Study Seven

Eight year old male with ASD, PDD diagnosed at age 3 along with dyspraxia, hypotonia, subclinical seizure disorder, suspected stroke-like episodes who presented with abnormal gait, poor coordination, left side weakness, left hand curls downward, nonverbal, sleeping difficulties, poor attention, brain fog, unresponsive to verbal stimuli, dysarthria, dysphonia, motor planning difficulties in gross and fine motor skills, cognitive deficits, learning problems, poor memory, social delay, tan stool, diarrhea, underweight, small for chronological age. During gestation mother consumed white albacore tuna daily and experienced gestational diabetes. Mother had a prior history of alcoholism. Paint had delays in developmental milestones, gained 100 words but lost speech at age two. Medications at time of consultation included Depakote 750 mg daily and Piracetam 2000 mg daily. Patient lives on a farm and has high exposure to pesticides and mold in home.

Our laboratory workup revealed a buildup of renegade fatty acids with suppression of both ω6 (GLA, DGLA, AA) and ω3 (ALA, EPA, DHA) fatty acids along with suppression of DMAs (myelin biomarkers) in the red cell fatty acid analysis, hyperammonemia, acidosis, increased LDH, electrolyte disturbance, low normal IGF-I (insulin growth factor reflective of methionine synthase function), positive for compound heterozygous MTHFR (methylene tetrahydrofolate reductase) mutations for C677T and A1298C, sharply elevated creatine kinase 228. Organic acid analysis revealed an increase in glutamic acid, citric acid, adipic acid and 5-hydroxyindoleacetic acid (5-HIAA) which may be linked to hepatic encephalopathy. Elevation of lead 120 (n=<15) after DMSA urinary challenge previously tested by another physician. (Absolutely no chemical chelators are used on children in our clinic). Patient had increased left side weakness after 13 months of oral DMSA was given.

Patient had a positive responsive to IV lipid exchange with 250 mg phosphatidylcholine followed by 0.3 cc leucovorin and 1200 mg GSH by IV fast push which was initially given once weekly along with targeted supplementation and nutrient dense diet. The dosing of the IV phosphatidylcholine was increased to 500 mg two to three times weekly and after 6 weeks there was dramatic improvement in response to others and the world around him, speech began to emerge with an explosion of complicated words, sleep improved, more social interaction—constantly trying to communicate. In essence the patient ‘awakened’ after liberal use of IV therapy. When the intravenous therapy was ceased for one month patient regressed in cognition, speech and coordination. Patient stabilized once intravenous therapy was re-introduced. Presently patient is making steady gains after bolus dosing of phosphatidylcholine which has resulted in increased language and awareness.

### Example 2

### Intravenous Administration of Pharmaneutical Compositions

a) Administration of PC Composition

A butterfly catheter with a 23-gauge needle was inserted into a vein of the antecubital region of one of the subjects' arms. A syringe containing the PC (phosphatidylcholine) composition in about 5 to about 10 cc volume was connected to the catheter by a flexible tube. A volume of blood equal to the total volume of the PC composition was drawn into the syringe and the syringe was gently agitated to mix the blood and PC composition. The blood/PC composition mixture was then infused (or “pushed”) as a lipid exchange into the subject over a period of two to three minutes.

b) Intravenous Administration of Leucovorin or Folinic Acid

A butterfly catheter with a 23-gauge needle was inserted into a vein of the antecubital region of one of the subjects' arms. The PC composition was infused first followed by a pre-prepared syringe containing about 2 mg (0.2 cc) to about 5 mg (0.5 cc) of Leucovorin over the period of 2-3 minutes.

c) Intravenous Administration of Reduced Glutathione

A butterfly catheter with a 23-gauge needle was inserted into a vein of the antecubital region of one of the subjects' arms. The PC and Leucovorin compositions were infused first followed by a pre-prepared syringe containing about 1.5 to 6 cc of glutathione generally pre-mixed with an equal portion of sterile water (not saline). The composition containing glutathione was followed the IV PC with a pre-prepared syringe of glutathione using the same needle. This procedure avoids re-sticking the patient by infusing first the PC, then the Leucovorin and then the glutathione using the same butterfly catheter with a flexible tube infused (or “pushed”) into the subject over a period of two to five minutes.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

### REFERENCES

Akasaka K, Shichijyukari S, Matsuoka S, Murata M, Meguro H, Ohrui H. Absolute configuration of a Ceramine with a novel Branched-Chain fatty acid isolated from the Epiphytic Dinoflagellate, Coolia monotis. Biosci Biotechnol Biochem September 2000; 64:9:842-1846
Akiba S, Sato T Cellular function of calcium-independent phospholipase A2 Biol Pharm Bull. 2004 August; 27:8:1174-8
Alesenko A V. The role of sphingomyelin cycle metabolites in transduction signals of cell proliferation, differentiation and death. Member Cell Biol. 2000; 13:2:303-20
Andrieu-Abadie N, Gouaze V, Salvarye R, Levade T. Ceramide in apoptosis signaling: relationship with oxidative stress. Free Radic Biol Med 2001; 31:717-728
Aoyama T, Souri M, Kamijo T, Ushikubo S, Hashimoto T. Peroxisomal Acyl-Coenzyme A Oxidase is a Rate-Limiting Enzyme in a Very-Long-Chain Fatty Acid β-Oxidation System. Biochemical and Biophysical Res Com Jun. 30, 1994; 201:3:1541-1547
Araki E, Kobayashi T, Kohtake N, Goto I, Hashimoto. A riboflavin-responsive lipid storage myopathy due to multiple acylCoA cehydrogenase deficiency: An adult case. J of the Neurological Sciences 1994; 126:202-205
Arrigoni E, Averet N, Cohadon F. Effects of CDP-choline on phospholipase A2 and cholinephosphotransferase activities following a cryogenic brain injury in the rabbit. Biochem Pharmacol. 1987 Nov. 1; 36:21:3697-700
Aschner M, Aschner J L. Mercury Neurotoxicity: Mechanisms of Blood-Brain Barrier Transport. Neurosci Biobehav Rev 1990 Summer; 14:169-176
Aschner M, Conklin D R, Yao C P, Allen J W, Tan K H. Induction of Astrocyte Metallothioneins by Zn confers resistance against the acute cytotoxic effects of Methylmercury on cell swelling, Na+ uptake and K+ release. Brain Research 1998; 813:254-261
Aschner M. Astrocytic swelling, phospholipase A2, glutathione and glutamate: interactions in methylmercury-induced neurotoxicity. Cell Mol Biol (Noisy-le-grand). 2000 June; 46:4:843-54
Aschner M, West A K. The role of MT in neurological disorders. J Alzheimers Dis. 2005 November; 8:2:139-45; discussion 209-15
Assies J, Haverkort E B, Lieverse R, Vreken P. Effect of dehydroepiandrosterone supplementation on fatty acid and hormone levels in patients with X-linked adrenoleucodystrophy. Clin Endocrinol (Oxf). 2003 October; 59:4:459-66
Attwell D, Miller B, Saantis M. Arachidonic acid as a messenger in the central nervous system. Seminars in the Neurosciences 1993; 5:159-169
Awasthi S, Vivekananda J, Awasthi V, Smith D, King R J. CTP:phosphocholine cytidylyltransferase inhibition by ceramide via PKC-alpha, p38 MAPK, cPLA2, and 5-lipoxygenase. Am J Physiol Lung Cell Mol Physiol 2001 July; 281:1:L108-18
Ballatori N, Truong A T. Cholestasis, altered junctional permeability, and inverse changes in sinusoidal and biliary glutathione release by vasopressin and epinephrine. Mol Pharmacol July 1990; 38:1:64-71
Barbosa F B, Capito K, Kofod H, Thams P. Pancreatic islet insulin secretion and metabolism in adult rats malnourished during neonatal life. Br J. Nutr. 2002 February; 87:2:147-55.
Barres B A, Raff M C. Proliferation of oligodendrocyte precursor cells depends on electrical activity in axons. Nature. 1993 Jan. 21; 361:6409:258-60
Bauman M L, Kemper T L The Neurobiology of autism Second Edition. Baltimore, Md. USA; The Johns Hopkins University Press, 1994, 2005
Bazan N G, Murphy M G, Toffano G. Neurobiology of Essential Fatty Acids. In: Advances in Experimental Medicine and Biology Vol 318 from the proceedings Jul. 10-12, 1991 Australia, New York: Plenum Publishing, 1992
Beaudet A L. Is medical genetics neglecting epigenetics? Genet Med. 2002 September-October; 4(5):399-402
Beier K, Volkl A Fahimi H D. Suppression of Peroxisomal Lipid β-oxidation enzymes by TNF-alpha. FEBS Lett 1992; 310:273-278
Bentley P, Calder I, Elcombe C, Grasso P, Stringer D, Wiegand H S. Hepatic peroxisome proliferation in rodents and its significance for humans. Food Chem Toxic 1993; 31:857 907
Bilak M, Wu L, Wang Q, Haughey N, Conant K, St Hillaire C, Andreasson K. PGE2 receptors rescue motor neurons in a model of amyotrophic lateral sclerosis. Ann Neurol. 2004 August; 56:2:240-8
Billis W, Fuks Z, Kolesnick R. Signaling in and regulation of ionizing radiation induced apoptosis in endothelial cells. Recent Prog Horm Res 1998; 53:85-92
Boadi W Y, Urbach J, Brandes J M, Yannai S. In vitro exposure to mercury and cadmium alters term human placental membrane fluidity. Toxicol Appl Pharmacol. 1992 September; 116:1:17-23
Boal D. Mechanics of a Cell, Boston: Cambridge University Press, 2002, chapter 1. p. 10
Bogdanovic M D, Kidd D, Briddon A, Duncan J S, Land J M. Late onset heterozygous ornithine transcarbamylase deficiency mimicking complex partial status epilepticus. J Neurol Neurosurg Psychiatry. 2000 December; 69:6:813-5
Boggs, K P, Rock C O, and Jackowski S. Lysophosphatidylcholine and 1-O-octadecyl-2-O-methyl-rac-glycero-3-phosphocholine inhibit the CDP-choline pathway of phosphatidylcholine synthesis at the CTP:phosphocholine cytidylyltransferase step. J Biol. Chem. 1995 Mar. 31; 270:13:7757-64
Bonacker D, Stoiber T, Wang M, Bohm K J, Prots I, Unger E, Thier R, Bolt H M, Degen G H. Genotoxicity of inorganic mercury salts based on disturbed microtubule function. Arch Toxicol. 2004 October; 78:10:575-83. Epub 2004 Jun. 15
Bourre J M, Francois M, Youyou A, Dumont O, Piciotti M, Pascal G, Durand G. The effects of dietary alpha-linolenic acid on the composition of nerve membranes, enzymatic activity, amplitude of electrophysiological parameters, resistance to poisons and performance of learning tasks in rats. J. Nutr. 1989 December; 119:12:1880-92
Brett J, Gerlach H, Nawroth P, Steinberg S, Godman G, Stern D. TNF/cachectin increases permeability of endothelial cell monolayers by a mechanism involving regulatory G proteins. J Exp Med 1989; 169: 1977-1991
Brown F R, Voight R, Singh A K, Singh I. Peroxisomal disorders. Neurodevelopmental and biochemical aspects. Am J Dis Child. 1993 June; 147:6:617-26
Brugg B, Michel P P, Agid Y, Ruberg M. Ceramide induces apoptosis in cultured mesencephalic neurons. J Neurochem 1996 February; 66:2:733-9
Brusilow S W, Finkelstien J. Restoration of nitrogen homeostasis in a man with ornithine transcarbamylase deficiency. Metabolism. 1993 October; 42:10:1336-9
Burlina A B, Ogier H, Korall H, Trefz F K. Long-term treatment with sodium phenylbutyrate in ornithine transcarbamylase-deficient patients. Eur J Paediatr Neurol. 2003; 7:3:115-21
Caruso L, Trischitta C, Bertino G, Amore M G, Rapisarda F, Calcara G. Polyunsaturated Pholsphatidylcholine in the treatment of Hepatic Steatosis. Clin Ter Nov. 30, 1983; 107:4:279-290
Cedrola S, Guzzi G, Ferrari D, Gritti A, Vescovi A L, Pendergrass J C, La Porta C A. Inorganic mercury changes the fate of murine CNS stem cells. FASEB J 2003 May; 17:8:869-71. Epub 2003 Mar. 28
Chang M C, Jones C R. Chronic lithium treatment decreases brain phospholipase A2 activity. Neurochem Res. 1998 June; 23:6:887-92
Chen A H, Innis S M, Davidson A G, James S J. Phosphatidylcholine and lysophosphatidylcholine excretion is increased in children with cystic fibrosis and is associated with plasma homocysteine, S-adenosylhomocysteine, and S-adenosylmethionine. Am J Clin Nutr. 2005 March; 81:3:686-91
Clark-Taylor T, Clark-Taylor B E. Is autism a disorder of fatty acid metabolism? Possible dysfunction of mitochondrial beta-oxidation by long chain acyl-CoA dehydrogenase. Med. Hypotheses. 2004; 62:6:970-5
Clayton P T. Clinical consequences of defects in peroxisomal beta oxidation Biochem Soc Trans 2001 May; 29:Pt 2:298-305
Cox C S, Dubey P, Raymond G V, Mahmood A, Moser A B, Moser H W. Cognitive evaluation of neurologically asymptomatic boys with X-linked adrenoleukodystrophy. Arch Neurol. 2006 January; 63:1:69-73
Crawford M, Marsh D. The Driving Force. 1989; NY, N.Y.: Harper and Row Publishers
Crawford M A, Costeloe K, Ghebremeskel K, Phylactos A, Skirvin L, Stacey F. Are deficits of arachidonic and docosahexaenoic acids responsible for the neural and vascular complications of preterm babies? Am J Clin Nutr. 1997 October; 66:4 Suppl:1032S-1041S
Chisolm J J Jr. Safety and efficacy of meso-2,3-dimercaptosuccinic acid (DMSA) in children with elevated blood lead concentrations. J Toxicol Clin Toxicol. 2000; 38:4:365-75
Cui A, Houweling M. Phosphatidylcholine and cell death. Biochim Biophys Acta 2002 Dec. 30; 1585:2-3:87-96
Cutler R G, Pedersen W A, Camandola S, Rothstein J D, Mattson M P. Evidence that accumulation of ceramides and cholesterol esters mediates oxidative stress-induced death of motor neurons in amyotrophic lateral sclerosis. Ann Neurol 2002 October: 52:4:448-57
Cutler R G, Kelly J, Storie K, Pedersen W A, Tammara A, Hatanpaa K, Troncoso J C, Mattson M P.
Involvement of oxidative stress-induced abnormalities in ceramide and cholesterol metabolism in brain aging and Alzheimer's disease. Proc Natl Acad Sci USA. 2004 Feb. 17; 101:7:2070-5
Daikhin Y, Yudkoff M. Ketone bodies and brain glutamate and GABA metabolism. Dev Neurosci 1998:20:358-364
Dasgupta S, Zhou Y, Jana M, Banik N L, Pahan K. Sodium phenylacetate inhibits adoptive transfer of experimental allergic encephalomyelitis in SJL/J mice at multiple steps. J. Immunol. 2003 Apr. 1; 170:7:3874-82
Davidson J, Abul H T, Milton A S, Rotondo D. Cytokines and Cytokine stimulate prostaglandin E2 entry into the brain. Pfugers Arch July 2001; 442:4:526-33
DeLeve L D, Kaplowitz N. Importance and regulation of hepatic glutathione. Semin Liver Dis November 1990; 10:4:251-66
Demirbilek S, Ersoy M O, Demirbilek S, Karaman A, Akin M, Bayraktar M, Bayraktar N. Effects of polyenylphosphatidylcholine on cytokines, nitrite/nitrate levels, antioxidant activity and lipid peroxidation in rats with sepsis. Intensive Care Med. 2004 October; 30(10):1974-8. Epub 2004 Mar. 26
Demirbilek S, Karaman A, Gurunluoglu K, Tas E, Akin M, Aksoy R T, Turkmen E, Edali M N, Baykarabulut A. Polyenylphosphatidylcholine pretreatment protects rat liver from ischemia/reperfusion injury. Hepatol Res. 2006
Dentico P., Volpe A., Buongiorno R., Grattagliano I., Altomare E., Tantimonaco G., Scotto G., Sacco R., Schiraldi O Glutathione in the treatment of chronic fatty liver diseases Recenti Prog Med 1995 July-August; 86:7-8:290-3
Deth R C. Molecular Origins of Human Attention: The Dopamine-Folate Connection. Norwell, Mass.: Kluwer Academic Publishers, 2003 Diczfalusy U. β-Oxidation of Eicosanoids. Prog Lipid Res 1994; 33:4:403-428
Di Santo E, Foddi M C, Ricciardi-Castagnoli P, Mennini T, Ghezzi P. DHEAS inhibits TNF production in monocytes, astrocytes and microglial cells. Neuroimmunomodulation September-October 1996; 3:5:285-8
Dunlop M, Clark S Glucose-induced phosphorylation and activation of a high molecular weight cytosolic phospholipase A2 in neonatal rat pancreatic islets Int J Biochem Cell Biol 1995 November; 27:11:1191-9
Dutczak W J, Clarkson T W, Ballatori N. Biliary-hepatic recycling of a xenobiotic gallbladder absorption of methylmercury. Am J Physiol 1991; 260: G873-G880 Ebadi M, Iversen P L, Hao R, Cerutis D R, Rojas P, Happe H K, Murrin L C, Pfeiffer R F. Expression and regulation of brain metallothionein. Neurochem Int July 1995; 27:1:1-22
Eisele K, Lang P A, Kempe D S, Klarl B A, Niemoller O, Wieder T, Huber S M, Duranton C, Lang F Stimulation of erythrocyte phosphatidylserine exposure by mercury ions Toxicol Appl Pharmacol. 2006 Jan. 1; 210:1-2:116-22
Farooqui A A, Horrocks L A. Excitatory amino acid receptors, neural membrane phospholipid metabolism and neurological disorders. Brain Res Brain Res Rev. 1991 May-August; 16:2:171-91
Farooqui A A, Litsky M L, Farooqui T, Horrocks L A. Inhibitors of intracellular phospholipase A2 activity: Their neurochemical effects and therapeutic importance of neurological disorders Brain Res Bull June 1999; 49:3:139-153
Farooqui A A, Ong W Y, Horrocks L A. Biochemical aspects of neurodegeneration in human brain: involvement of neural membrane phospholipids and phospholipases A2. Neurochem Res. 2004 November; 29:11:1961-77
Femandez-Checa J C, Yi J R, Ruiz C G, Ookhtens M, Kaplowitz N. Plasma membrane and mitochondrial transport of hepatic reduced glutathione. Seminars in Liver Disease 1996; 16:2:147-158
Fitsanakis V A, Aschner M. The importance of glutamate, glycine, and gamma-aminobutyric acid transport and regulation in manganese, mercury and lead neurotoxicity. Toxicol Appl Pharmacol. 2005 May 1; 204:3:343-54
Fortenberry J D, Owens M L, Chen N X, Brown L A. S-nitrosoglutathione inhibits TNF-alpha-induced NFkappaB activation in neutrophils. Inflamm Res. 2001 February; 50:2:89-95
Foster J. S., Kane P C, Speight N. The Detoxx Book: Detoxification of Biotoxins in Chronic Neurotoxic Syndromes. Millville, N.J. USA: BodyBio, 2002
Fourcade S, Savary S, Gondcaille C, Berger J, Netik A, Cadepond F, El Etr M, Molzer B, Bugaut M Thyroid hormone induction of the adrenoleukodystrophy-related gene (ABCD2). Mol. Pharmacol. 2003 June; 63(6):1296-303
Fusunyan R D, Quinn J J, Ohno Y, MacDermott R P, Sanderson I R. Butyrate enhances interleukin (IL)-8 secretion by intestinal epithelial cells in response to IL-1beta and lipopolysaccharide. Pediatr Res. 1998 January; 43:1:84-90
Garcia J J, Martinez-Ballarin E, Millan-Plano S, Allue J L, Albendea C, Fuentes L, Escanero J F. Effects of trace elements on membrane fluidity. J Trace Elem Med Biol. 2005; 19:1:19-22
Gardian G, Yang L, Cleren C, Calingasan N Y, Klivenyi P, Beal M F Neuroprotective effects of phenylbutyrate against MPTP neurotoxicity Neuromolecular Med. 2004; 5:3:235-41
Gibson G G, Milton M N, Elcombe C R Induction of Cytochrome P450 IVA 1-Mediated Fatty Acid. Hydroxylation: Relevance to Peroxisome Profileration Biochemical Society Transactions 1990; 18:97-99
Gibson G G, Lake B Peroxisomes: Biology and Importance in Toxicology and Medicine London Taylor and Francis, 1993
Goldfarb R D, Parker T S, Levine D M, Glock D, Akhter I, Alkhudari A, McCarthy RJ, David E M, Gordon B R, Saal S D, Rubin A L, Trenholme G M, Parrillo J E. Protein-free phospholipid emulsion treatment improved cardiopulmonary function and survival in porcine sepsis. Am J Physiol Regul Integr Comp Physiol. 2003 Gondcaille C, Depreter M, Fourcade S, Lecca M R, Leclercq S, Martin P G, Pineau T, Cadepond F, ElEtr M, Bertrand N, Beley A, Duclos S, De Craemer D, Roels F, Savary S, Bugaut M. Phenylbutyrate up-regulates the adrenoleukodystrophy-related gene as a nonclassical peroxisome proliferator. J. Cell Biol. 2005 Apr. 11; 169:1:93-104. Epub 2005 Apr. 4
Gordon B R, Parker T S, Levine D M, Saal S D, Hudgins L C, Sloan B J, Chu C, Stenzel K H, Rubin A L. Safety and pharmacokinetics of an endotoxin-binding phospholipid emulsion. Ann Pharmacother. 2003 July-August; 37:7-8:943-50
Gordon B R, Parker T S, Levine D M, Feuerbach F, Saal S D, Sloan B J, Chu C, Stenzel K H, Parrillo J E, Rubin A L. Neutralization of endotoxin by a phospholipid emulsion in healthy volunteers. J Infect Dis. 2005
Grandjean P, Weihe P. Arachidonic acid status during pregnancy is associated with polychlorinated biphenyl exposure. Am J Clin Nutr. 2003 March; 77:3:715-9
Greenfield E A, Reddy J, Lees A, Dyer C A, Koul O, Nguyen K, Bell S, Kassam N, Hinojoza J, Eaton M J, Lees M B, Kuchroo V K, Sobel R A Monoclonal antibodies to distinct regions of human myelin proteolipid protein simultaneously recognize central nervous system myelin and neurons of many vertebrate species J Neurosci Res 2006 Feb. 15; 83:3:415-31
Gu M, Kerwin J L, Watts J D, Aebersold R. Ceramide profiling of complex lipid mixtures by electrospray ionization mass spectrometry. Anal Biochem 1997; 244:347-356
Gurr M L, Harwood J L, Frayn K N. Lipid Biochemistry An Introduction 5^{th} Edition 2002; Malden, M A: Blackwell Science, Inc.
Guengerich F P. Reactions and significance of cytochrome P-450 enzymes. J Biol Chem 1991 Jun. 5; 266:16:10019-22. Review
Gutknecht J. Inorganic Mercury (Hg2+) Transport through the Lipid Bilayer Membranes. J Membrane Biol 1981; 61: 61-66
Haddad J J, Harb H L. L-gamma-Glutamyl-L-cysteinyl-glycine (glutathione; GSH) and GSH-related enzymes in the regulation of pro- and anti-inflammatory cytokines: a signaling transcriptional scenario for redox(y) immunologic sensor(s)? Mol. Immunol. 2005 May; 42:9:987-1014. Epub 2004 Nov. 23
Hameroff S, Nip A, Porter M, Tuszynski J. Conduction pathways in microtubules, biological quantum computation, and consciousness. Biosystems. 2002 January; 64:1-3:149-68
Haughey N J, Cutler R G, Tamara A, McArthur J C, Vargas D L, Pardo C A, Turchan J, Nath A, Mattson M P. Perturbation of sphingolipid metabolism and ceramide production in HIV-dementia. Ann Neurol 2004 February; 55:2:257-67
Hayashi H, Takahata S. Role of peroxisomal fatty acyl-CoA beta-oxidation in phospholipid biosynthesis. Arch Biochem Biophys. 1991 Feb. 1; 284:2:326-31
Hayter H L, Pettus B J, Ito F, Obeid L M, Hannun Y A. TNFalpha-induced glutathione depletion lies downstream of cPLA2 in L929 cells. FEBS Letts Oct. 2, 2001; 507:2:151-6
Herbert M R, Ziegler D A, Deutsch C K, O'Brien L M, Lange N, Bakardjiev A, Hodgson J, Adrien K T, Steele S, Makris N, Kennedy D, Harris G J, Caviness V S Jr. Dissociations of cerebral cortex, subcortical and cerebral white matter volumes in autistic boys. Brain. 2003a May; 126:Pt 5:1182-92
Herbert M R, Ziegler D A, Deutsch C K, Makris N, Bakardjiev A, Hodgson J, Adrien K T. Larger brain and white matter volumes in children with developmental language disorder. Dev Sci 2003b; 6:F11-22
Herbert M R Large brains in autism: the challenge of pervasive abnormality. Neuroscientist. 2005 October; 11:5:417-40
Hofmann K, Tomiuk S, Wolff G, Stoffel W. Cloning and Characterization of the Mammalian Brain-Specific Mg2+-Dependant Neutral Sphingomyelinase. Proc Natl Acad Sci USA 2000; 97:5895-5900
Horning M, Lipkin W I. Infectious and immune factors in the pathogenesis of neurodevelopmental disorders: epidemiology, hypothesis, and animal models. Ment Retard Dev Disabil Res Rev 2001; 7:200-10
Horning M, Chian D, Lipkin W I. Neurotoxic effects of postnatal thimerosal are mouse strain dependent. Mol Psychiatry 2004; 9:833-45
Horrobin D F, Jenkins K, Bennett C N, Christie W W. Eicosapentaenoic acid and arachidonic acid: collaboration and not antagonism is the key to biological understanding. Prostaglandins Leukot Essent Fatty Acids. 2002 January; 66:1:83-90.
Howard, S, Chan-Yeung M, Martin L, Phaneuf S, and Salari H. Polyphosphoinositide hydrolysis and protein kinase C activation in guinea pig tracheal smooth muscle cells in culture by leukotriene D4 involve a pertussis toxin sensitive G-protein. Eur J. Pharmacol. 1992 Oct. 1; 227:2:123-9
Hresko R C, Sugar I P, Barenholz Y, Thompson T E. The lateral distribution of pyrene-labeled sphingomyelin and gluceosylceramide in phosphatidylchoine bilayers. Biophys J 1987 May; 51:5: 725-33
Jaeschke H, Wemer C, Wendel A. Disposition and hepatoprotection by phosphatidylcholine liposomes in mouse liver. Chem Biol Interact 1987; 64:1-2:127-137
Jakobs B S, Wanders R J A. Conclusive evidence that very-long-chain fatty acids are oxidized exclusively in peroxisomes in human skin fibroblasts. Biochem Biophys Res Commun 1991 Aug. 15; 178:3:842-7
Jakobs B S, Wanders R J A. Fatty acid β-oxidation in peroxisomes and mitochondria: the first, unequivocal evidence for the involvement of carnitine in shuttling propionyl-CoA from peroxisomes to mitochondria. Biochem Biophys Res Com Aug. 24, 1995; 213:3:1035-1041
James S J, Cutler P, Melnyk S, Jernigan S, Janak L, Gaylor D W, Neubrander J A. Metabolic biomarkers of increased oxidative stress and impaired methylation capacity in children with autism. Am J Clin Nutr. 2004 December; 80:6:1611-7
James S J, Slikker W 3rd, Melnyk S, New E, Pogribna M, Jernigan S. Thimerosal neurotoxicity is associated with glutathione depletion: protection with glutathione precursors. Neurotoxicology. 2005 January; 26:1:1-8
Jenkins P J, Portmann B P, Eddleston A L, Williams R. Use of polyunsaturated phosphatidyl choline in HBsAg negative chronic active hepatitis: results of prospective double-blind controlled trial. Liver 1982 June; 2:2:77-81
Johnson J K, Kumar N R, Srivastava D K. Molecular basis of the medium-chain fatty acyl-CoA dehydrogenase-catalyzed “oxidase” reaction: pH-dependent distribution of intermediary enzyme species during catalysis. Biochemistry. 1994 Apr. 19; 33:15:4738-44
Kane P C. Peroxisomal Disturbances in Children with Epilepsy, Hypoxia and Autism. Prostaglandins, Leukotrienes and Essential Fatty Acids August 1997a; 57:2:265
Kane P C, Kane E. Peroxisomal Disturbances in Autistic Spectrum Disorder. J Ortho Med; 1997b; 12:4:207-218
Kane P C. The Neurobiology of Lipids in Autistic Spectrum Disorder J Ortho Med 1999; 14:2:103-109
Kane P C, Foster J S, Cartaxo A. Clinical detoxification of neurotoxins and heavy metals in autistic spectrum disorder. Autism, Genes and the Environment UMDNJ, October 2002a
Kashireddy P V, Rao M S. Lack of peroxisome proliferator-activated receptor alpha in mice enhances methionine and choline deficient diet-induced steatohepatitis. Hepatol Res. 2004 October; 30:2:104-110
Keller F, Persico A M. The neurobiological context of autism. Mol. Neurobiol. 2003 August; 28:1:1-22
Kemp S, Wei H M, Lu J F, Braiterman L T, McGuinness M C, Moser A B, Watkins P A, Smith K D. Gene redundancy and pharmacological gene therapy: implications for X-linked adrenoleukodystrophy. Nat Med 1998 November; 4:11:1261-8
Kerper L E, Mokrzan E M, Clarkson T W, Ballatori N. Methylmercury efflux from brain capillary endothelial cells is modulated by intracellular glutathione but not ATP. Toxicol Appl Pharmacol. 1996 December; 141:2:526-31
Kinnunen P K, Holopainen J M Sphingomyelinase activity of LDL: a link between atherosclerosis, ceramide, and apoptosis? Trends Cardiovasc Med. 2002 January; 12:1:37-42
Kitchens R L, Wolfbauer G, Albers J J, Munford R S. Plasma lipoproteins promote the release of bacterial lipopolysaccharide from the monocyte cell surface. J Biol. Chem. 1999 Nov. 26; 274(48):34116-22
Klein N J, Shennan G I, Heyderman R S, Levin M. Alteration in glycosaminoglycan metabolism and surface charge on human umbilical vein endothelial cells induced by cytokines, endotoxin and neutrophils. J Cell Sci. 1992 August; 102: Pt 4:821-32
Kodama K, Suzuki M, Toyosawa T, Araki S. Inhibitory mechanisms of highly purified vitamin B2 on the productions of proinflammatory cytokine and NO in endotoxin-induced shock in mice. Life Sci. 2005 Nov. 26; 78:2:134-9. Epub 2005 Aug. 19
Kohl O Myelin and autism Paper presented at the International Meeting for Autism Research, 2001, San Diego
Kramer B C, Yabut J A, Cheong J, Jnobaptiste R, Robakis T, Olanow C W, Mytilineou C. Toxicity of glutathione depletion in mesencephalic cultures: a role for arachidonic acid and its lipoxygenase metabolites. Eur J. Neurosci. 2004 January; 19:2:280-6
Kronke M. Involvement of shingomylinases in TNF signaling pathways. Chem Phys Lipids. 1999 November; 102:1-2:157-66
Kunau W H, Dommes V, Schulz H. β-Oxidation of Fatty Acids in Mitochondria, Peroxisomes and Bacteria: A Century of Continued Progress. Prog Lipid Res 1995; 34:4:267-342
Kyllerman M, Blomstrand S, Mansson J E, Conradi N G, Hindmarsh T. Central nervous system malformations and white matter changes in pseudo-neonatal adrenoleukodystrophy. Neuropediatrics. 1990 November; 21:4:199-201
Lageweg W, Tager J M, Wanders R J A. Topography of VLCFA activating activity in peroxisomes from rat liver. Biochem J. 1991 May 15; 276: Pt 1:53-6
Latruffe N, Cherkaoui Malki M, Nicolas-Frances V, Jannin B, Clemencet M C, Hansmannel F, Passilly-Degrace P, Berlot J P. Peroxisome-proliferator-activated receptors as physiological sensors of fatty acid metabolism: molecular regulation in peroxisomes. Biochem Soc Trans. 2001 May; 29:Pt 2:305-9
Lazo O, Contreras M, Singh I. Topographical Localization of Peroxisomal Acyl-CoA Ligases: Differential Localization of Palmitoyl-CoA and Lignoceroyl-CoA Ligases. Biochemistry 1990a; 29:3981-3986
Lazo O, Contreras M, Yoshida Y, Singh A K, Stanley W, Weise M, Singh Y. Cellular Oxidation of Lignoceric Acid is Regulated by the Subcellular Localization of Palmitoyl-CoA and Lignoceroyl-CoA Ligases Biochemistry 1990b; 29:3981-3986
Leiper J M, Birdsey G M, Oatey P B. Peroxisomes Proliferate. Trends in Cell Biol November 1995; 5:435-437
Leite M, Frizzo J K, Nardin P, de Almeida L M, Tramontina F, Gottfried C, Goncalves C A. Beta-hydroxy-butyrate alters the extracellular content of S100B in astrocyte cultures. Brain Res Bull. 2004 Aug. 30; 64:2:139-43
Lewine J D, Andrews R, Chez M, Patil A A, Devinsky O, Smith M, Kanner A, Davis J T, Funke M, Jones G, Chong B, Provencal S, Weisend M, Lee R R, Orrison W W Jr. Magnetoencephalographic patterns of epileptiform activity in children with regressive autism spectrum disorders. Pediatrics. 1999 September; 104:3 Pt 1:405-18
London E A. The environment as an etiologic factor in autism: a new direction for research. Environ Health Perspect. 2000 June; 108 Suppl 3:401-4
Luers G, Beier K, Hashimoto T, Fahimi H D, Volkl A. Biogenesis of peroxisomes: sequential biosynthesis of the membrane and matrix proteins in the course of hepatic regeneration. Eur J Cell Biol 1990 August; 52:2:175-84
Luquet S, Lopez-Soriano J, Holst D, Gaudel C, Jehl-Pietri C, Fredenrich A, Grimaldi P A. Roles of peroxisome proliferator-activated receptor delta (PPARdelta) in the control of fatty acid catabolism. A new target for the treatment of metabolic syndrome. Biochimie. 2004 November; 86(11):833-7
Maestri N E, Brusilow S W, Clissold D B, Bassett S S. Long-term treatment of girls with ornithine transcarbamylase deficiency. N Engl J. Med. 1996 Sep. 19; 335:12:855-9
Mandel H, Berant M, Aizin A, Gershony R, Hemmli S, Schutgens R B, Wanders R J. Zellweger-like phenotype in two siblings: a defect in peroxisomal β-oxidation with elevated very long-chain fatty acids but normal bile acids. J Inherit Metab Dis 1992; 15:3:381-4
Mandel H, Espeel M, Roels F, Sofer N, Luder A, Iancu T C, Aizin A, Berant M, Wanders R J, Schutgens R B. A new type of peroxisomal disorder with variable expression in liver and fibroblasts. J Pediatr 1994 October; 125:4:549-55
Mannaerts G P, Van Veldhoven P P. Role of peroxisomes in mammalian metabolism. Cell Biochem Funct 1992 September; 10:3:141-51 Review
Marchi B, Burlando B, Moore M N, Viarengo A. Mercury- and copper-induced lysosomal membrane destabilisation depends on [Ca2+]i dependent phospholipase A2 activation Aquat Toxicol. 2004 Feb. 10; 66:2:197-204
MacDonell L E, Skinner F K, Ward P E, Glen A I, Glen A C, Macdonald D J, Boyle R M, Horrobin D F. Increased levels of cytosolic phospholipase A2 in dyslexics. Prostaglandins Leukot Essent Fatty Acids. 2000 July-August; 63:1-2:37-9
McGiff J C. Cytochrome P-450 metabolism of arachidonic acid. Annu Rev Pharmacol Toxicol 1991; 31:339-69. Review
McGuinness M C, Moser A B, Poll—The BT, Watkins P P A. Complementation analysis of patients with intact peroxisomes and impaired peroxisomal beta-oxidation. Biochem Med Metab Biol 1993 April; 49:2:228-42
Metz S A. Is phospholipase A2 a “glucose sensor” responsible for the phasic pattern of insulin release? Prostaglandins. 1984 January; 27:1:147-58. Miles A T, Hawksworth G M, Beattie J H, Rodilla V. Induction, regulation, degradation, and biological significance of mammalian metallothioneins. Crit. Rev Biochem Mol Biol 2000; 35:1:35-70
Minamiyama M, Katsuno M, Adachi H, Waza M, Sang C, Kobayashi Y, Tanaka F, Doyu M, Inukai A, Sobue G. Sodium butyrate ameliorates phenotypic expression in a transgenic mouse model of spinal and bulbar muscular atrophy. Hum Mol. Genet. 2004 Jun. 1; 13:11:1183-92
Minshew N J, Goldstein G, Dombrowski S M, Panchalingam K, Pettegrew J W. A preliminary 31P MRS study of autism: evidence for undersynthesis and increased degradation of brain membranes. Biol Psychiatry 1993; 33:262-73
Moscona-Amir E, Henis Y I, Yechiel E, Barenholz Y, Sokolovsky M. Role of lipids in age-related changes in the properties of muscarinic receptors in cultured rat heart myocytes. Biochemistry 1986 Dec. 2; 25:24:8118-24
Moser H W, Raymond G V, Dubey P. Adrenoleukodystrophy: new approaches to a neurodegenerative disease. JAMA. 2005a Dec. 28; 294:24:3131-4
Moser H W, Raymond G V, Lu S E, Muenz L R, Moser A B, Xu J, Jones R O, Loes D J, Melhem E R, Dubey P, Bezman L, Brereton N H, Odone A. Follow-up of 89 asymptomatic patients with adrenoleukodystrophy treated with Lorenzo's oil Arch Neurol. 2005b July; 62:7:1073-80.
Moser, A B, Kreiter N, Bezman L, Lu S, Raymond G V, Naidu S, Moser H W. Plasma very long chain fatty acids in 3,000 peroxisome disease patients and 29,000 controls. Ann Neurol. 1999 January; 45:1:100-10
Moser H W, Moser A B. Peroxisomal disorders: overview. Ann N Y Acad. Sci. 1996a Dec. 27; 804:427-41. Review
Moser H W, Moser A B. Very long-chain fatty acids in diagnosis, pathogenesis, and therapy of peroxisomal disorders. Lipids. 1996b March; 31 Suppl:S141-4
Moser A B, Rasmussen M, Naidu S, Watkins P A, McGuinness M, Hajra A K, Chen G, Raymond G, Liu A, Gordon D, et al. Phenotype of Patients with Peroxisomal Disorders Subdivided into Sixteen Complementation Groups. J of Pediatr July 1995; 127:1:13-22
Mostofsky D I, Yehuda S, Rabinovitz S, Carasso R L. The control of blepharospasm by essential fatty acids. Neuropsychobiology 2000; 41:3:154-7
Mouritsen O G. Life—As a matter of fat, the emerging science of lipidomics. Berlin Heidelberg, Germany: Springer-Verlag, 2005
Nagai H, Matsumaru K, Feng G, Kaplowitz N. Reduced glutathione depletion causes necrosis and sensitization to tumor necrosis factor-alpha-induced apoptosis in cultured mouse hepatocytes. Hepatology July 2002; 36:1:55-64
Naito Y, Konishi C, Ohora N. Blood Coagulation and Osmolar Tolerance of Erythrocytes in Stroke-Prone spontaneously Hypertensive rats given rapeseed oil or soybean oil as the only dietary fat. Toxicology Letters 2000; 116:3:209-215
Nordberg M, Nordberg G F. Toxicological Aspects of Metallothionein. Cell Mol Biol (Noisy-le-grand). 2000 March; 46:2:451-63. Review
Ogawa M, Sato N, Endo S, Kojika M, Yaegashi Y, Kimura Y, Ikeda K, Iwaya T Group IIA-soluble phospholipase A2 levels in patients with infections after esophageal cancer surgery Surg Today. 2005; 35:11:912-8
Osuchowski M F, Edwards G L, Sharma R P. Fumonisin B1-induced neurodegeneration in mice after intracerebroventricular infusion is concurrent with disruption of sphingolipid metabolism and activation of proinflammatory signaling. Neurotoxicology. 2005 March; 26:2:211-21
Pahan K, Sheikh F G, Namboodiri A M, Singh I. Lovastatin and phenylacetate inhibit the induction of nitric oxide synthase and cytokines in rat primary astrocytes, microglia, and macrophages. J Clin Invest. 1997 Dec. 1; 100(11):2671-9
Pardo C A, Vargas D L, Zimmerman A W. Immunity, neuroglia and neuroinflammation in autism. Int Rev Psychiatry. 2006 January; 17:6:485-95
Peet M, Glen I, Horrobin D F. Phospholipid Spectrum Disorder in Psychiatry. Carnforth, Lancashire, UK: Marius Press 1999
Pendergrass J C, Haley B E, Vimy M J, Winfield S A, Lorscheider F L Mercury vapor inhalation inhibits binding of GTP to tubulin in rat brain: similarity to a molecular lesion in Alzheimer diseased brain. Neurotoxicology. 1997; 18:2:315-24
Pena L F, Hill D B, McClain C J. Treatment with glutathione precursor decreases cytokine activity. JPEN J Parenter Enteral Nutr January-February 1999; 23:1:1-6
Petri S, Kiaei M, Kipiani K, Chen J, Calingasan N Y, Crow J P, Beal M F. Additive neuroprotective effects of a histone deacetylase inhibitor and a catalytic antioxidant in a transgenic mouse model of amyotrophic lateral sclerosis. Neurobiol Dis. 2005 Nov. 10.
Petroni A. Androgens and fatty acid metabolism in X-linked Adrenoleukodystrophy. Prostaglandins Leukot Essent Fatty Acids. 2002 August-September; 67:2-3:137-9
Poggi-Travert F, Fournier B, Poll—The BT, Saudubray J M. Clinical approach to inherited peroxisomal disorders. J Inherit Metab Dis. 1995; 18 Suppl 1:1-18 Review
Pogribny I P, Ross S A, Wise C, Pogribna M, Jones E A, Tryndyak V P, James S J, Dragan Y P, Poirier L A. Irreversible global DNA hypomethylation as a key step in hepatocarcinogenesis induced by dietary methyl deficiency. Mutat Res. 2005 Sep. 3.
Poll—The BT, Roels F, Ogier H, Scotto J, Vamecq J, Schutgens R B, Wanders R J, van Roermund C W, van Wijland M J, Schram A W, et al. A new peroxisomal disorder with enlarged peroxisomes and a specific deficiency of acyl-CoA oxidase (pseudo-neonatal adrenoleukodystrophy). Am J Hum Genet. 1988 March; 42:3:422-34.
Porter T D, Coon M J. Cytochrome P450 Multiplicity: Isoforms, Substrates and Catalytic and Regulatory Mechanisms. Journ of Biol Chem 1991; 266:13469
Praphanphoj V, Boyadjiev S A, Waber L J, Brusilow S W, Geraghty M T. Three cases of intravenous sodium benzoate and sodium phenylacetate toxicity occurring in the treatment of acute hyperammonaemia.
J Inherit Metab Dis. 2000 March; 23:2:129-36
Qi X, Hosoi T, Okuma Y, Kaneko M, Nomura Y. Sodium 4-phenylbutyrate protects against cerebral ischemic injury. Mol. Pharmacol. 2004 October; 66:4:899-908.
Rachubinski R A, Subramani S. How proteins penetrate peroxisomes. Cell November 17; 1995; 83:525-528
Ram P A, Waxman D J. DHEA 3 β-sulfate is an endogenous activator of the peroxisome-proliferation pathway: Induction of cytochrome P450 4A and acyl-Co oxidase mRNAs in primary rat hepatocyte culture and inhibitory effects of Ca++ channel blockers. Biochem J 1994 Aug. 1; 301: Pt 3:753-8
Rao M S, Ide H, Alvares K, Subbarao V, Reddy J K, Hechter O, Yeldandi A V. Comparative effects of dehydroepiandrosterone and related steroids on peroxisome proliferation in rat liver. Life Sci. 1993; 52(21):1709-16
Rapoport S I. In vivo labeling of brain phospholipids by long-chain fatty acids: relation to turnover and function. Lipids 1996; 31:S97-S101
Rapoport S I. In vivo fatty acid incorporation into brain phospholipids in relation to signal transduction and membrane remodeling. Neurochem Res 1999 November; 24:11:1403-15
Reddy J K. Peroxisomal Lipid Metabolism. Annu Rev Nutr 1994; 14:343-70
Rediske, J, Morrissey M M, and Jarvis M. Human monocytes respond to leukotriene B4 with a transient increase in cytosolic calcium. Cell Immunol. 1993 Apr. 1; 147:2:438-45
Rodilla V, Miles A T, Jenner W, Hawksworth G M. Exposure of cultured human proximal tubular cells to cadmium, mercury, zinc and bismuth: toxicity and metallothionein induction. Chem Biol Interact. 1998 Aug. 14; 115:1:71-83.
Roels F, Espeel M, Poggi F, Mandel H, Van Maldergem L, Saudubray J M. Human Liver Pathology in Peroxisomal Diseases: A Review including Novel Data. Biochimie 1993; 75:281-292
Rubenstein J L, Merzenich M M. Model of autism: increased ratio of excitation/inhibition in key neural systems. Genes Brain Behav 2003; 2:255-67
Rudin D O. Unpublished manuscript: The Omega Factor: Our Nutritional Missing Link, 1985, Chapter 7. p. 18
Rustan A C, Christiansen E N, Drevon A C. Serum lipids, Hepatic Glycerolipid Metabolism and Peroxisomal fatty acid oxidation in rats fed omega-3 and omega-6 fatty acids. Biochem J 1992; 283:333-339
Ryu H, Smith K, Camelo S I, Carreras I, Lee J, Iglesias A H, Dangond F, Cormier K A, Cudkowicz M E, Brown R H Jr, Ferrante R J. Sodium phenylbutyrate prolongs survival and regulates expression of anti-apoptotic genes in transgenic amyotrophic lateral sclerosis mice. J. Neurochem. 2005 June; 93:5:1087-98
Sato M, Sasaki M, Oguro T, Kuroiwa Y, Yoshida T. Induction of Metallothionein Synthesis by Glutathione depletion after trans- and cis-stilbene oxide administration in rats. Chemico-Biological Interactions 1995; 98:15-25
Schachter D., Abbott R E, Cogan U, Flamm M. Lipid fluidity of the individual hemileaflets of human erythrocyte membranes. Ann N Y Acad Sci 1983; 414:19-28
Segain J P, Bletiere D R, Bourreille A, Leray V, Gervois N, Rosales C, Ferrier L, Bonnet C, Blottiere H M, Galmiche J P. Butyrate inhibits Inflammatory responses through NFkB inhibition: Implications for Crohn's Disease. Gut 2000; 47:397-403
Shanker G, Hampson R E, Aschner M. Methylmercury stimulates arachidonic acid release and cytosolic phospholipase A2 expression in primary neuronal cultures. Neurotoxicology. 2004 March; 25:3:399-406
Shanker G, Syversen T, Aschner M. Astrocyte-mediated methylmercury neurotoxicity. Biol Trace Elem Res. 2003 October; 95:1:1-10
Sharma A, Waly M, Deth R C. Protein kinase C regulates dopamine D4 receptor-mediated phospholipid methylation. Eur J Pharmacol 2001 Sep. 14; 427:2:83-90
Sharma R, Lake B G, Foster J, Gibson G G. Microsomal Cytochrome P452 Induction and Peroxisomal Proliferation by Hypolipidaemic Agents in Rat Liver: A Mechanistic Inter-Relationship. Biochem Pharm 1988; 37:1193-1201
Sharma A, Kramer M L, Wick P F, Liu D, Chari S, Shim S, Tan W, Ouellette D, Nagata M, DuRand C J, Kotb M, Deth R C. D4 dopamine receptor-mediated phospholipid methylation and its implications for mental illnesses such as schizophrenia. Mol. Psychiatry. 1999 May; 43:235-46
Shibutani T, Johnson T M, Yu Z X, Ferrans V J, Moss J, Epstein S E Pertussis toxin-sensitive G proteins as mediators of the signal transduction pathways activated by cytomegalovirus infection of smooth muscle cells
J Clin Invest 1997 Oct. 15; 100:8:2054-61
Shrago E, Woldegiorgis G, Ruoho A E, DiRusso C C Fatty Acyl CoA esters as regulators of cell metabolism. Prostaglandins Leukot Essent Fatty Acids 1995 February-March; 52:2-3:163-6. Review
Shrief M K, Thompson E J In vivo relationship of TNF-alpha to BBB damage in patients with active MS. J Neuroimmunol 1993; 38:27-34
Simon D K, Rodriguez M L, Frosch M P, Quackenbush E J, Feske S K, Natowicz M R. A unique familial leukodystrophy with adult onset dementia and abnormal glycolipid storage: a new lysosomal disease? J Neurol Neurosurg Psychiatry. 1998 August; 65:2:251-4
Singh H, Poulos A. Distinct Long Chain and very long chain fatty acyl-CoA Synthetases in Rat Liver Peroxisomes and Microsomes. Archives of Biochemistry and Biophysics 1988; 266:486-495
Singh I, Moser A E, Goldfischer S, Moser H W. Lignoceric Acid is Oxidized in the Peroxisome: Implications for the Zellweger Cerebro-hepato-renal Syndrome and ALD. Proc Nat Acad Sci 1984; 81:4203-4207
Sokol D K, Kunn D W, Edwards-Brown M, Feinberg J. Hydrogen proton magnetic resonance spectroscopy in autism: preliminary evidence of elevated choline/creatinine ratio. J Child Neurol 2002; 17:245-9
Stoiber T, Bonacker D, Bohm K J, Bolt H M, Thier R, Degen G H, Unger E. Disturbed microtubule function and induction of micronuclei by chelate complexes of mercury(II). Mutat Res. 2004 Oct. 10; 563:2:97-106
Sun G Y, Hu Z Y Stimulation of phospholipase A2 expression in rat cultured astrocytes by LPS, TNF alpha and IL-1 beta Prog Brain Res 1995; 105:231-8
Susanto I, Wright S E, Lawson R S, Williams C E, Deneke S M. Metallothionein, glutathione, and cystine transport in pulmonary artery endothelial cells and NIH/3T3 cells. Am J Physiol February 1998; 274:2 Pt 1:L296-300
Thies F, Nebe-von-Caron G, Powell J R, Yaqoob P, Newsholme E A, Calder P C. Dietary supplementation with eicosapentaenoic acid, but not with other long-chain n-3 or n-6 polyunsaturated fatty acids, decreases natural killer cell activity in healthy subjects aged >55 y. Am J Clin Nutr. 2001 March; 73:3:539-48
Toyosawa T, Suzuki M, Kodama K, Araki S. Effects of intravenous infusion of highly purified vitamin B2 on lipopolysaccharide-induced shock and bacterial infection in mice. Eur J. Pharmacol. 2004 May 25; 492:2-3:273-80
Tserng K Y, Chen L S and Jin S J. Comparison of metabolic fluxes of cis-5-enoyl-CoA and saturated acyl-CoA through the β-oxidation pathway. Biochem J 1995; 307:23-28
Tsukamoto T, Ishikawa M, Yamamoto T. Suppressive effects of TNF-alpha on myelin formation in vitro. Acta Neurol Scan January 1995; 91:1:71-5
Van den Bosch H, Schutgens R B H, Wanders R J A, Tager J. Biochemistry of the Peroxisomes. Annu Rev Biochem 1992; 61:157-197.
Vanden Heuvel J P, Sterchele P F, Nesbit D J, Peterson R E. Coordinate induction of acyl-CoA binding protein, fatty acid binding protein and peroxisomal beta-oxidation by peroxisome proliferators. Biochim Biophys Acta 1993 Jun. 6; 1177:2:183-90
van Geel B M, Assies J, Wanders R J, Barth P G. X linked adrenoleukodystrophy: clinical presentation, diagnosis, and therapy. J Neurol Neurosurg Psychiatry. 1997 July; 63:1:4-14
Van Maldergem L, Espeel M, Wanders R J, Roels F, Gerard P, Scalais E, Mannaerts G P, Casteels M, Gillerot Y. Neonatal seizures and severe hypotonia in a male infant suffering from a defect in peroxisomal β-oxidation. Neuromuscul Disord 1992; 2:3:217-24
van Velhoven P P, Vanhove G, Asselberghs S, Eyssen H J, Mannaerts G P. Substrate specificities of rat liver peroxisomal acyl-CoA oxidases: Palmitoyl-CoA oxidase (inducible acyl-Co oxidase), pristanoyl-CoA oxidase (non-inducible acyl-CoA oxidase) and trihydroxycoprotanoyl-CoA oxidase. J Biol Chem 1992; 267:20065-20074
Vargus D L, Nascimbene C, Krishnan C, Zimmerman A W, Pardo C A. Neuroglial activation and neuroinflammation in the brain of patients with autism. Ann Neurol 2005; 57:67-81
Verity M A, Sarafian T, Pacifici E H K, Seranian A. Phospholipase A2 Stimulation by Methyl Mercury in Neuron culture. J of Neurochem 1994; 62:705-714
Vivekananda J, Smith D, King R J. Sphingomyelin metabolytes inhibit sphingomyelin synthase and CTP:phosphocholine cytidyltransferase. Am J Physiol Lung Cell Mol Physiol 2001; 281:L98-L107.
Walton P A, Hill P E, Subramani S. Import of Stably Folded Proteins into Peroxisomes. Molecular Biology of the Cell 1995 June; 6:675-683
Waly M, Olteanu H, Banerjee R, Choi S W, Mason J B, Parker B S, Sukumar S, Shim S, Sharma A, Benzecry J M, Power-Chamitsky V A, Deth R C. Activation of methionine synthase by insulin-like growth factor-1 and dopamine: a target for neurodevelopmental toxins and thimerosal. Mol. Psychiatry. 2004 April; 9:4:358-70
Wanders R J. Peroxisomes, lipid metabolism, and peroxisomal disorders. Mol Genet Metab. 2004 September-October; 83:1-2:16-27
Wanders R J, van Roermund C W, van Wijland M J, Schutgens R B, van den Bosch H, Schram A W, Tager J M. Direct demonstration that the deficient oxidation of VLCFA in x-linked ALD due to an impaired ability to activate VLCFA. Biochem Biophys Res Commun 1998; 153:618-624
Wanders R J, Heymans H S, Schutgens R B, Barth P G, van den Bosch H, Tager J M. Peroxisomal disorders in neurology. J Neurol Sci. 1988 December; 88:1-3:1-39
Wang W, Ballatori N. Endogenous glutathione conjugates: occurrence and biological functions. Pharmacol Rev. 1998 September; 50:3:335-56
Watanabe H, Shimojo N, Sano K, Yamaguchi S. The distribution of total mercury in the brain after the lateral ventricular singe injection of methylmercury and glutathione. Res Commun Chem Pathol Pharmacol 1988 April; 60:1: 57-69
Watkins P A, McGuinness M C, Raymond G V, Hicks B A, Sisk J M, Moser A B, Moser H W. Distinction between peroxisomal bifunctional enzyme acyl-CoA oxidase deficiencies. Ann Neurol 1995 September; 38:3:472-7
Wei H, Kemp S, McGuinness M C, Moser A B, Smith K D. Pharmacological induction of peroxisomes in peroxisome biogenesis disorders. Ann Neurol. 2000 March; 47:3:286-96
Weihe P Grandjean P, Jorgensen P J. Application of hair-mercury analysis to determine the impact of a seafood advisory. Environ Res. 2005 February; 97:2:200-7
Woodman R J, Mori T A, Burke V, Puddey I B, Watts G F, Beilin L J. Effects of purified eicosapentaenoic and docosahexaenoic acids on glycemic control, blood pressure, and serum lipids in type 2 diabetic patients with treated hypertension. Am J Clin Nutr. 2002 November; 76:5:1007-15
Wu A, Hinds C J, Thiemermann C High-density lipoproteins in sepsis and septic shock: metabolism, actions, and therapeutic applications Shock. 2004 March; 21:3:210-21
Xu L, Ash M, Abdel-aleem S, Lowe J E, Badr M. Hyperinsulinemia inhibits hepatic peroxisomal beta-oxidation in rats. Horm Metab Res 1995 February; 27:2:76-8
Yechiel E, Barenholz Y. Relationships between membrane lipid composition and biological properties of rat myocytes. Effects of aging and manipulation of lipid composition. J Biol. Chem. 1985a Aug. 5; 260:16:9123-31
Yechiel E, Barenholz Y, Henis Y. Lateral Mobility and Organization of Phospholipids and Protein in Rat Myocyte membranes. J Biol Chem 1985b; 260:9132-9136
Yechiel E, Barenholz Y. Cultured heart cell aggregates: a model for studying relationships between aging and lipid composition. Biochim Biophys Acta 1986 Jul. 10; 859:1:105-9
Yehuda S, Carasso R L. Modulation of learning pain thresholds, and thermoregulation in the rat by preparations of free-purified alpha linolenic and linoleic acids: determination of the optimal w3-to-w6 ratio. Proc Natl Acad Sci USA 1993; 90:10345-10349
Yehuda S, Carasso R L, Mostofsky D I. Essential fatty acid preparation (SR-3) raises the seizure threshold in rats. Eur J Pharmacol 1994 Mar. 11; 254:1-2:193-8
Yeon J E, Choi K M, Baik S H, Kim K O, Lim H J, Park K R, Kim J Y, Park J J, Kim J S, Bak Y T, Byun K S, Lee C H. Reduced expression of peroxisome proliferator-activated receptor-alpha may have an important role in the development of non-alcoholic fatty liver disease. J Gastroenterol Hepatol. 2004 July; 19:7:799-804
Yiin S J, Lin T H. Effects of metallic antioxidants on cadmium-catalyzed peroxidation of arachidonic acid. Ann Clin Lab Sci. 1998 January-February; 28:1:43-50.
Yin L, Laevsky G, Giardina C. Butyrate suppression of colonocyte NF-kappa B activation and cellular proteasome activity. J Biol Chem November 2001; 276:48:44641-6
Yu Z, Nikolova-Karakashian M, Zhou D, Cheng G, Schuchman E H, Mattson M P. Pivotal role for acidic sphingomyelinase in cerebral ischemia-induced ceramide and cytokine production, and neuronal apoptosis. J Mol Neurosci 2000 October; 15:2:85-97
Yudkoff M, Daikhin Y, Nissim I, Lazarow A, Nissim I. Brain amino acid metabolism and ketosis. J Neurosci Res 2001 Oct. 15; 66:2:272-81
Zalups R K, Barfuss D W. Accumulation and Handling of Inorganic Mercury in the Kidney after Coadministration with Glutathione. J Toxicol Environ Health 1995; 44:385-399
Zalups R K, Barfuss D W, Lash L H. Disposition of inorganic mercury following biliary obstruction and chemically-induced glutathione depletion: Dispositional changes 1 h after the intravenous administration of mercuric chloride. Toxicol Appl Pharmacol 1999a; 115:135-144
Zalups R K, Barfuss D W, Lash L H. Relationships between alterations in glutathione metabolism and the disposition of inorganic mercury in rats: Effects of biliary ligation and chemically induced modulation of glutathione status. Chem Biol Interact 1999b; 123:171-195
Zhao R, Chen Y, Tan W, Waly M, Sharma A, Stover P, Rosowsky A, Malewicz B, Deth R C. Relationship between dopamine-stimulated phospholipid methylation and the single-carbon folate pathway. J. Neurochem. 2001 August; 78:4:788-96
Zweigner J, Gramm H J, Singer O C, Wegscheider K, Schumann R R. High concentrations of lipopolysaccharide-binding protein in serum of patients with severe sepsis or septic shock inhibit the lipopolysaccharide response in human monocytes. Blood. 2001 Dec. 15; 98:13:3800-8
Zweigner J, Jackowski S, Smith S H, Van Der Merwe M, Weber J R, Tuomanen E I. Bacterial inhibition of phosphatidylcholine synthesis triggers apoptosis in the brain. J Exp Med. 2004 Jul. 5; 200:1:99-106
Zheng W, Aschner M, Ghersi-Egea J F. Brain barrier systems: a new frontier in metal neurotoxicology research. Toxicol Appl Pharmacol. 2003 Oct. 1; 192:1:1-11. Review

What is claimed is:

1. A pharmaneutical composition for treating, preventing, or ameliorating the symptoms of fatty acids imbalance and cell membrane dysfunction in an individual comprising an effective amount of a first and a second composition, the first composition comprises one or more phosphatidylcholine formulations and the second composition comprises one or more constituents comprising essential fatty acid supplements, trace minerals, phenylbutyrate, electrolytes, methylating agents, glutathione, or a combination thereof, in a suitable carrier.

2. The pharmaneutical composition of claim 1, further comprising peroxisomal cocktails including thiamin, riboflavin, pyridoxine, biotin, pantothenic acid, NADH, carnitine, CoQ10, or a combination thereof.

3. The pharmaneutical composition of claim 1, wherein the first composition, the second composition, or both are formulated in one solution.

4. The pharmaneutical composition of claim 1, wherein the first composition, the second composition, or both are formulated in different solutions.

5. The pharmaneutical composition of claim 1, wherein the first composition, the second composition, or both are administered contemporaneously.

6. The pharmaneutical composition of claim 1, wherein the first composition, the second composition, or both are administered at different time intervals.

7. The pharmaneutical composition of claim 1, wherein the first composition, the second composition, or both are administered in a time-released manner.

8. The pharmaneutical composition of claim 1, wherein the first composition, the second composition, or both are in a dry formulation.

9. The pharmaneutical composition of claim 7, wherein the first composition, the second composition, or both are in a liquid formulation.

10. The pharmaneutical composition of claim 1, wherein the essential fatty acid supplements comprise linoleic acid and alpha linolenic acid in a ratio of about 4:1.

11. The pharmaceutical composition of claim 1, wherein the methylating agents comprise vitamin B compounds.

12. The pharmaneutical composition of claim 10, wherein the vitamin B compounds comprise B12, and B complex compounds.

13. The pharmaceutical composition of claim 11, wherein the B12 and B complex compounds comprise Methylcobalamin, and folinic acid compounds comprising Leucovorin, Citrovorum and, Wellcovorin, or a combination thereof.

14. The pharmaneutical composition of claim 1, wherein the trace minerals comprise E-Lyte Liquid Mineral™ set #1-8 containing separate solutions of biologically available potassium, zinc, magnesium, copper, chromium, manganese, molybdenum, and selenium, or a combination thereof.

15. The pharmaneutical composition of claim 1, wherein the electrolytes comprise sodium, potassium, chloride, calcium, magnesium, bicarbonate, phosphate, and sulfate, or a combination thereof.

16. A method of treating, ameliorating, or preventing the symptoms of diseases and disorders related to imbalance of fatty acids and cell membrane dysfunction in a in a subject comprising administering to the subject an effective amount of a pharmanuetical composition comprising a first and a second composition, the first composition comprising one or more phosphotidylcholine formulations and the second composition comprises one or more constituents comprising essential fatty acid supplements, trace minerals, butyrate, electrolytes, methylating agents, glutathione, or a combination thereof, in a suitable carrier, wherein the subject is treated or the symptoms of the diseases and disorders in the subject are treated, ameliorated, or prevented.

17. The method of claim 16, wherein the diseases and disorders comprises autism.

18. The method of claim 16, wherein the first composition, the second composition, or both are administered intravenously, orally, or both.

19. The method of claim 17, wherein the pharmaneutical composition is administered through the following steps:
i) intravenous administration of a first phosphatidylcholine composition comprising about 500 mg to 1000 mg phosphatidylcholine, followed by intravenous administration leucovorin of about 5 mg to about 10 mg, and followed by about 1800 mg to about 2400 mg of reduced glutathione, twice daily for 3 to 5 days in a seven-day period;
ii) once daily oral administration of a second phosphatidylcholine composition comprising about 3600 to about 18,000 mg of phosphatidylcholine daily;
iii) once or twice daily oral administration of an effective amount of one or more trace minerals;
iv) five times daily oral administration of electrolytes;
v) once or twice daily oral administration of about 30 mls to about 60 mls of an EFA 4:1 composition;
vi) once or twice daily oral administration of about 910 mg to about 2600 mg gamma linolenic acid as evening primrose oil;
vii) once or twice daily oral or intravenous administration of an effective amount of one or more vitamin B complex compositions, Leucovorin/Folinic acid; and
viii) once daily oral, sublingual, or injectable administration of an effective amount of one or more Methylcobalamin compositions,
wherein the subject is treated or the symptoms of autism in the subject are ameliorated, or prevented.

20. A kit for the treatment, amelioration, or prevention of the symptoms of diseases and disorders related to fatty acids imbalance and cell membrane dysfunction in a subject, comprising:
a) a first composition comprising one or more phosphatidylcholine formulations;
b) a second composition comprising one or more constituents comprising:
i) linoleic acid and alpha linolenic acid in a ratio of about 4:1;
ii) trace minerals;
iii) butyrate or phenylbutyrate;
iv) electrolytes;
v) methylating agents; and
vi) glutathione,
c) instructions for the use of the first and second compositions; and
d) instructions for where to obtain any missing components of the kit.
